(19) United States
(12) Patent Application Publication
(10) Pub. No.: US 2025/0281743 A1
(43) Pub. Date: Sep. 11, 2025

(54) Transcutaneous Electrical Spinal Cord Stimulator for Treating Pain

(71) Applicants: The United States Government as represented by the Department of Veterans Affairs, Washington, DC (US); The Regents of the University of California, Oakland, CA (US)

(72) Inventors: Anastasia Keller, San Francisco, CA (US); Jeannie Bailey, San Francisco, CA (US); Adam Ferguson, San Francisco, CA (US); Prasad Shirvalkar, San Francisco, CA (US)

(21) Appl. No.: 18/859,621

(22) PCT Filed: Apr. 25, 2023
(86) PCT No.: PCT/US2023/019793

§ 371 Date: Oct. 24, 2024

Related U.S. Application Data
(60) Provisional application No. 63/334,432, filed on Apr. 25, 2022.

(51) Int. Cl. A61N 1/36; G16H 20/30

(52) U.S. Cl. CPC A61N 1/36021; A61N 1/36017; A61N 1/36031; A61N 1/36034; G16H 20/30

(57) ABSTRACT

Disclosed herein is a method of treating pain, such as, for example, chronic lower back pain. One or more electrodes can be positioned proximate to a spinal cord. Using the one or more electrodes, a transdermal stimulation routine can be applied to a portion of the spinal cord to treat pain.

### CROSS-REFERENCE TO RELATED APPLICATION

This application claims priority to and the benefit of the filing date of U.S. Provisional Patent Application No. 63/334,432, filed Apr. 25, 2022, the entirety of which, including the appendices, is hereby incorporated by reference herein.

### FIELD

This disclosure relates to systems and methods for treating pain, such as, for example, chronic lower back pain, with use of electrical stimulation.

### BACKGROUND

As a leading cause of disability worldwide, chronic low back pain (cLBP) represents a significant medical and socioeconomic problem with estimated health care spending of $87 billion annually. Patients with pain such as cLBP have limited treatment alternatives. The current standard of care including conservative physical therapy, cognitive behavioral therapy, nonsteroidal anti-inflammatory medications as well as invasive surgical approaches have only marginal therapeutic efficacy based on patient reported outcomes. In many situations, patients are limited largely to pharmaceuticals such as opioids, which have negative side effects and do not directly promote long-term improvement.

Accordingly, a need exists for a better treatment for pain such as chronic lower back pain.

### SUMMARY

Disclosed herein, in one aspect, is a method of treating pain. One or more electrodes can be positioned proximate to a spinal cord. Using the one or more electrodes, a transdermal stimulation routine can be applied to a portion of the spinal cord to treat pain.

Disclosed herein is a method of treating pain in a patient. The method comprises applying a transdermal stimulation routine to a portion of a spinal cord to treat pain.

Disclosed herein is a method of determining a transdermal stimulation routine for treating pain in a patient. The method comprises changing at least one of a pulse duration, a frequency, or an amplitude of the transdermal stimulation routine based on a pain metric.

Disclosed herein is a method of treating pain in a patient. The method comprises applying, transdermally, by a first electrode, a stimulation routine to a portion of a spinal cord and increasing an intensity of the stimulation routine to a first threshold.

Also disclosed herein is a method including performing physical therapy during or after receipt of transdermal stimulation routine to a portion of a spinal cord to treat pain while experiencing reduced pain from the transdermal stimulation routine.

Additional advantages of the disclosed apparatuses, systems, and methods will be set forth in part in the description that follows, and in part will be obvious from the description, or may be learned by practice of the claimed invention. The advantages of the disclosed devices and systems will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

These and other features of the preferred embodiments of the invention will become more apparent in the detailed description in which reference is made to the appended drawings wherein:

FIG. 1 is a schematic diagram showing implantable electrodes for stimulating a spinal cord as is known in the art.

FIG. 2 illustrates placement of electrodes for transcutaneous spinal cord stimulation (tSCS).

FIG. 3 is a schematic drawing showing stimulation of a spine and mechanisms triggered in the spine.

FIG. 4 is an illustration of a stimulation routine not shown to scale in order to show different parameters.

FIG. 5 shows an exemplary stimulation routine at different time scales.

FIG. 6A shows exemplary signal generators that are Food and Drug Administration-approved. FIG. 6B shows an exemplary signal generator for performing a tSCS method as disclosed herein.

FIG. 7 illustrates schematic flow of electric field through a spine.

FIGS. 8A-8E illustrate different methods for performing functional assessments of a patient. FIG. 8A shows schematics of biomechanical analysis. FIG. 8B shows brain fMRI scans, showing functional and structural assessments. FIG. 8C shows data from a paraspinal SEMG. FIG. 8D shows a lumbar MRI, showing muscle quality. FIG. 8E shows a neurophysiological assessment of motor-evoked potentials, indicating spinal cord excitability.

FIG. 9 illustrates tSCS+ABT safely improve trunk control in a child with spinal cord injury Segmental trunk joint kinematics (A), center of pressured (COP) displacement (B) and T10 (C) and L5 (D) Erector Spinae (ES) sEMG during anterior leaning task at baseline vs. post 40 training session.

FIG. 10 shows an EEG obtained during systematic variation of SCS programs. (A) Left-Xray of Thoracic Spine with implanted SCS electrodes to treat lumbosacral pain, Right-Patient drawing of leg pain area shaded black. (B-E) Overhead scalp heatmaps of EEG activity. Baseline beta-band neural activity is strongest over right frontal lobe (B, red=higher power), when pain is rated 7/10 similar to ineffective (C) or subthreshold SCS (D). With optimal SCS parameters that reduce pain to 4/10, (E) beta activity is highest over left frontal cortex. However, individual single frequency or brain region maps do not generalize across patients.

FIG. 11 shows the M13 module correlates highest to spinal cord injury (SCI) severity (Spearman rho=0.82). On the top left is the heatmap of the top-seeded genes for this module, and on the bottom left is the eigengene score for each one of the patients (and controls) for this module. The line plot on the right shows the expression levels of the top 15 genes of the M13 module across all 58 samples. (color scheme in x-axis labels in panel B is as follows: blue=HC, green=TC, brown=AIS ‘D’, purple=AIS ‘C’, salmon=AIS ‘B’, and red=AIS ‘A’).

FIG. 12 shows a receiver operating characteristic plots for the AIS ‘A’ against the remaining SCIs (left) and the AIS ‘D’ against the remaining SCIs (right). These plots show the strong predictive ability of the model for SCI patients with AIS ‘A’ and ‘D’. The AUC is 0.865 for the ‘A’ and 0.938 for the ‘D’. N=12 ‘A’ vs. 21 SCIs and 11 ‘D’ vs. 22 SCIs.

FIG. 13 shows pain reduction following each treatment session for a first patient.

FIG. 14 shows pain reduction following each treatment session for a second patient.

FIG. 15 shows aggregated data for pain reduction for a plurality of patients (n=4) after a plurality of treatment sessions.

FIG. 16 is a schematic diagram of a system comprising a stimulator, a computing device for controlling an output of the stimulator, and electrodes in communication with the stimulator.

FIG. 17 shows a block diagram of a system for using machine learning to evaluate pain indication metrics.

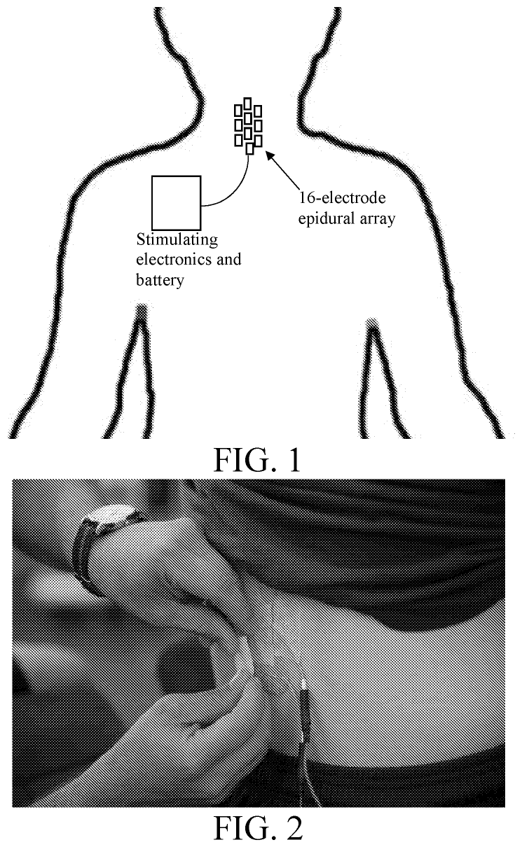

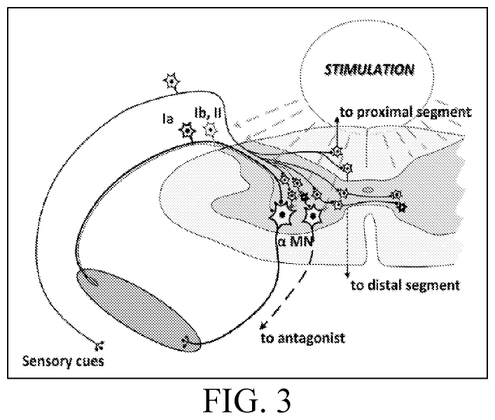

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “an electrode” can refer to one or more of such electrodes, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

The word “or” as used herein means any one member of a particular list and, unless context dictates otherwise, can, in alternative aspects, also include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

As used herein, “pain” can be defined as an unpleasant sensory and emotional experience associated with actual or potential tissue damage, or described in terms of such damage. In some aspects, pain treated according to systems and methods disclosed herein can include unpleasant sensory and emotional experience in the absence of stimulus (e.g., phantom limb pain). In other aspects, pain treated according to systems and methods disclosed herein can be limited to pain in response to stimulus.

As used herein, a “spinal cord injury” can be defined as damage to any part of the spinal cord or nerves at the end of the spinal canal (cauda equina). In some aspects, spinal cord injury can cause permanent changes in strength, sensation and other body functions below the site of the injury.

As used herein, the term “patient” can refer to a human or animal subject who is experiencing pain. Optionally, in exemplary aspects, the “patient” can refer to a human or animal subject who does not have a spinal cord injury. Optionally, in exemplary aspects, the “patient” can refer to a human or animal subject who has not been diagnosed with a spinal cord injury.

### BACKGROUND

To date, spinal cord stimulation (SCS) for treatment of cLBP has been delivered via epidural electrodes. Although recent clinical trials on such SCS show promising outcomes in patients with cLBP, these SCS procedures require neurosurgical implantation with the inherent risk of post-surgical complications.

Transcutaneous SCS (tSCS) provides a non-invasive neuromodulation technique in the field of spinal cord injury used to potentiate lumbosacral spinal cord excitability to enable improvement in motor functions. However, tSCS has not been used for treatment of pain (e.g., pain associated with cLBP), in part due to the intensity required being prohibitively high. Such high intensities are known to be painful or harmful to the skin.

Transcutaneous spinal cord stimulation as further disclosed herein is a novel, safe, and highly efficacious approach to treat cLBP with the advantage of being non-invasive, thus finding a wider use/accessibility for patients with cLBP. It is contemplated that the disclosed tSCS systems and methods can open avenues for further device development/advancement as well as initiate new investigations of tSCS to treat other neuromuscular pain conditions.

### Transdermal SCS for Treating Pain

Disclosed herein, in various aspects, is a method of treating pain in a patient. The method comprises applying a transdermal stimulation routine to a portion (or portions) of a spinal cord to treat pain. In some aspects, the patient does not have a spinal cord injury that affects motor function (and, optionally, has not been diagnosed with a spinal cord injury that affects motor function). Optionally, in further aspects, the patient does not have any spinal cord injury (and, optionally, has not been diagnosed with any spinal cord injury). Optionally, in other aspects, the patient can have a spinal cord injury, and the transdermal stimulation routine can be applied using stimulation parameters (e.g., repetition frequencies) that are effective to treat pain but that are not effective to treat spinal cord injury. For example, in these aspects, it is contemplated that the disclosed transdermal stimulation routine can be effective to reduce pain but be ineffective at achieving/potentiating motor function.

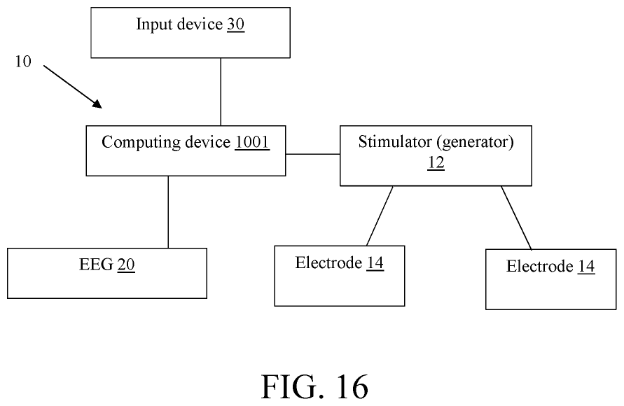

Electrodes 14 (e.g., cathodes) (FIG. 16) can be placed proximate to specific vertebrae. For example, to treat lower back pain, electrodes can be placed at one or more of T8, T10, L1 vertebrae for spinal cord stimulation. The number of stimulating electrodes can be iteratively determined through stimulation optimization sessions. The stimulation optimization sessions can take place in an initial series of therapy sessions (e.g., the first 2-3 therapy sessions) and can use patient feedback regarding the amount of pain relief achieved (e.g., comparing results using different numbers of electrodes, for example, results using 2 electrodes compared with results using 3 electrodes, and/or the respective positions of the electrodes). One or more electrodes of opposed polarity (e.g., anodes) can be placed bilaterally over the iliac crests and/or paraumbilically (over the abdomen). In some aspects, electrodes (e.g., cathodes) can be placed proximate to the cervical spine (e.g., at C5 and/or C2). One or more electrodes of opposed polarity (e.g., anodes) can be placed bilaterally or directly opposite said electrodes proximate to the cervical spine (e.g., over the clavicle bones). Although specific arrangements of the electrodes and cathodes are discussed above, it should be understood that the positions of electrodes and cathodes can be reversed.

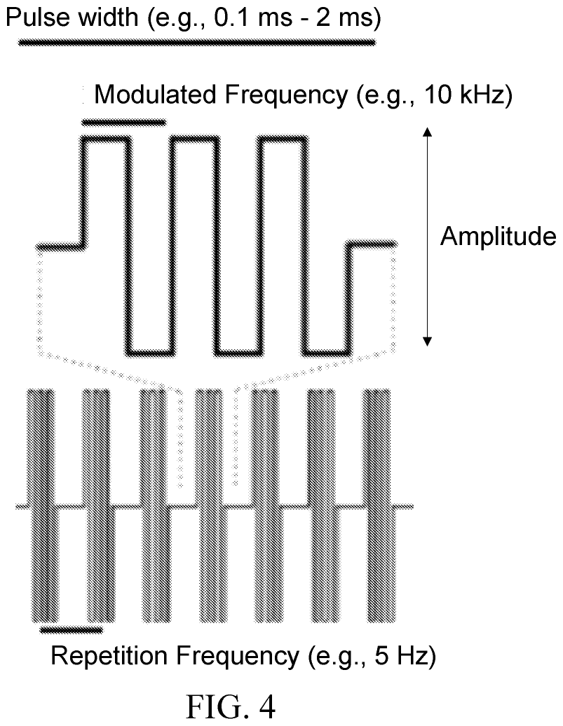

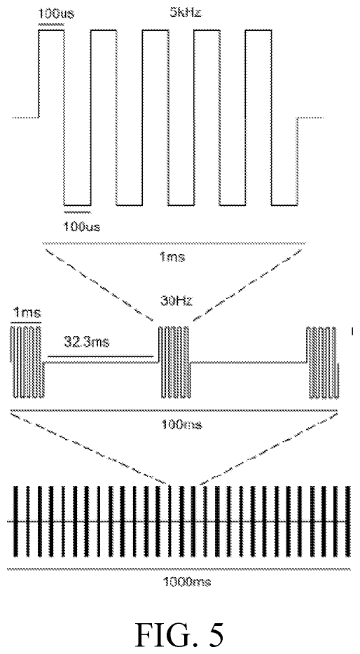

Referring to FIG. 4, FIG. 5, and FIG. 16, an exemplary stimulator (generator) 12 can be configured to generate a series of pulses to provide a stimulation routine. The stimulation routine can comprise a plurality of repetitions (e.g., pulse sequences). Each repetition can have a pulse width. Each repetition can comprise a plurality of frequency modulated pulses provided at a carrier frequency. The carrier frequency can inhibit discomfort resulting from the stimulation routine. The repetitions can be repeated at a repetition frequency.

The stimulation routine can have an intensity. In some aspects, the intensity can be a function of amplitude. In some aspects, the intensity can further be a function of pulse width. Neuronal activation can be a function of phase charge, which can be the product of (current) amplitude and pulse duration. Phase charge can determine stimulus strength and can effect motor fiber recruitment and torque generation. In some aspects, the intensity can further be a function of repetition frequency. Generally, the stimulation intensity delivered for pain treatment can be patient-specific and, in exemplary aspects, can be dependent on the patient's tolerance/sensory thresholds of stimulation intensity. In some aspects, the intensity can be higher than the intensity used for enabling certain movements in adults with spinal cord injury. For example, some patients experiencing pain may be able to tolerate a high intensity of stimulation, as compared to those intensities required to enable certain movements in adults with spinal cord injury. Thus, in some aspects, some patients experiencing pain can receive transcutaneous pain therapy at a stimulation intensity that exceeds the stimulation intensity associated with treatment of spinal cord injury. In some exemplary aspects, pain relief can be dependent on the stimulation intensity.

The stimulation routine and/or electrode positioning for treating pain can differ from a stimulation routine configured to treat SCI. For example, to achieve/potentiate motor function in individuals with SCI, researchers use a 30 Hz repetition frequency at the T10 electrode and a 15 Hz or 30 Hz repetition frequency at the L1 electrode. In exemplary aspects, to treat pain, a repetition frequency of 45 Hz at electrodes placed at T8, T10 and L1 can be provided. More broadly, in some exemplary aspects, it is contemplated that the repetition frequency used to treat pain can be at least 40 Hz, at least 45 Hz, or at least 50 Hz.

In some aspects, the stimulation routine can be monophasic. In other aspects, the stimulation routine can be biphasic.

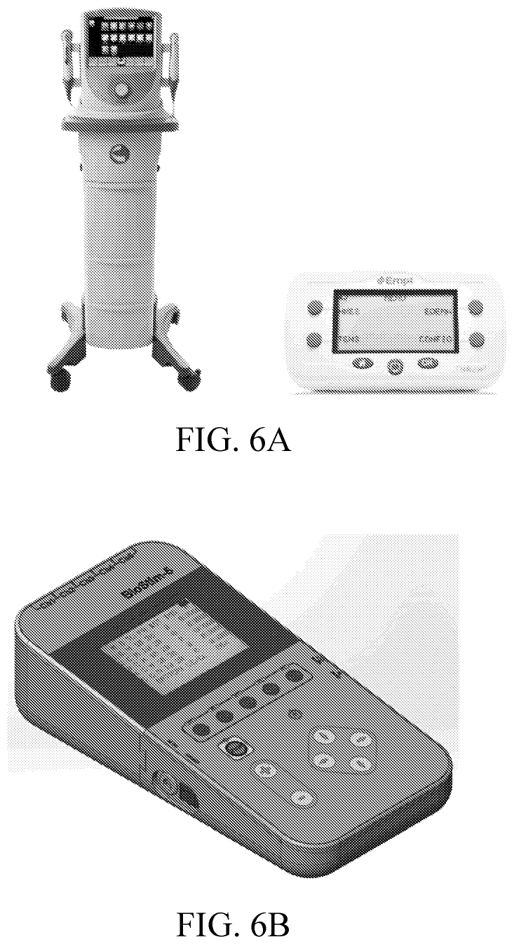

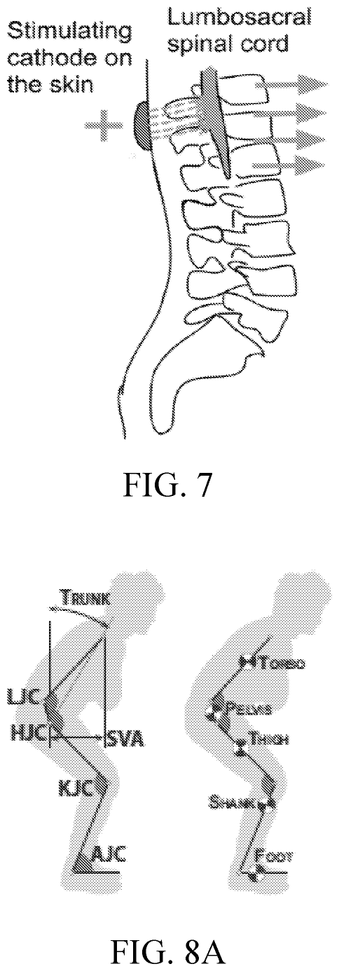

FIG. 6A illustrate exemplary stimulators (generators) that are FDA approved, including the Vectra Neo (Chattanooga, Hixton, TN) and Empi Continuum, DJO Global, (Vista, CA, USA). FIG. 6B illustrates an exemplary stimulator (generator), BioStim-5 (Cosyma, LTD, Russia), used in examples described below.

In some aspects, the transdermal stimulation routine can be tuned for a particular patient in order to achieve optimal pain reduction. A method of determining a transdermal stimulation routine for treating pain in a patient can comprise changing at least one of a pulse duration, a repetition frequency, a carrier frequency, or an amplitude of the transdermal stimulation routine based on a pain metric. For example, the transdermal stimulation routine can be changed from an initial starting setting based on the pain metric (e.g., a patient reported pain metric).

In some aspects, the pain metric can be a subjective patient opinion. Optionally, a patient can enter pain metric information through a user interface. Alternatively, it is contemplated that a clinician can enter pain metric information into a user interface based upon feedback provided by the patient to the clinician.

In some aspects, the pain metric can be based on or correspond to an EEG measurement. For example, the pain metric can be based on or correspond to a determined change in brain activity (as reflected in the EEG), measured using either the whole brain spectral power density or localized to specific brain regions of interest, such as those known to process pain related sensory information.

In some aspects, the pain metric can be a biomarker. For example, the biomarker can be ribonucleic acid (RNA), such as RNA from blood cells. Further disclosure of RNA biomarkers indicative of pain as well as methods for isolating relevant RNA/specific genes and gene cluster biomarkers are disclosed in the Examples provided herein.

Referring to FIGS. 4 and 5, in various aspects, the transdermal stimulation routine can have a pulse duration (pulse width) from 0.1 ms to 2 ms. In some aspects, the pulse duration can be from 2 ms to 5 ms. In some aspects, the pulse duration can be from 5 ms to 20 ms, or longer than 20 ms. In various aspects, the transdermal stimulation routine can have an amplitude from 0 to 250 mA. In some aspects, the transdermal stimulation routine can have an amplitude from 0 to 1000 mA. In some aspects, the transdermal stimulation routine can have an amplitude from 275 mA to 1000 mA. It is contemplated that higher body fat can require a greater amplitude. In various aspects, the transdermal stimulation routine can have a pulse repetition frequency from 1 Hz to 99 Hz (e.g., optionally, from about 30 Hz to about 45 Hz). In some aspects, the transdermal stimulation routine can have a pulse repetition frequency from 1 Hz to 1000 Hz. In various aspects, the transdermal stimulation routine can have a modulated frequency from 2 kHz to 10 KHz. In some aspects, the transdermal stimulation routine can have a modulated frequency from 4 kHz to 20 KHz.

In some aspects, a machine learning algorithm can be used to determine or modify the transdermal stimulation routine for treating pain. For example, the at least one of a frequency or the amplitude can be changed (e.g., from the initial starting settings) based on a machine learning algorithm. In some aspects, the machine learning algorithm can comprise a support vector machine. For example, in some aspects, data from stimulation routines and results (e.g., one or more biomarkers and/or patient subjective opinion) can be provided to a machine learning module, and the machine-learning module can analyze the data to determine an optimal stimulation routine.

Referring to FIG. 16, in some aspects, a computing device 1001 can be in communication with an EEG 20 or other brain scanning apparatus. The computing device can be in communication with, and configured to control, a stimulator (generator) 12 that generates the transdermal stimulation routine. The computing device can be configured to vary the transdermal stimulation routine based on data from EEG or other brain scanning apparatus. The computing device 1001 can be, for example, a standalone computing device, such as a personal computer or tablet. In other aspects, the computing device 1001 can be embodied as a microprocessor. For example, in some aspects, the computing device can be integral to the stimulator 12.

In some aspects, the computing device 1001 can apply a machine learning algorithm to optimize the transdermal stimulation routine based on a pain state predicted based on the data from the EEG or other brain scanning apparatus. In some aspects, the computing device can apply a machine learning algorithm to predict a pain state based on data from the EEG or other brain scanning apparatus.

In some aspects, the method can further comprise moving at least one electrode from a first location on the patient to a second location on the patient. The repositioning of electrodes may be a part of the stimulation optimization to best target individual pain relief.

In some aspects, the stimulation routine can be determined at least in part as a function of the etiology of the pain. For example, neuropathic pain and myofascial pain can be treated with different stimulation routines.

In still further aspects, it is contemplated that a patient can be determined to be responsive or non-responsive to a stimulation routine. For example, in some aspects, the patient can be determined to be responsive or non-responsive based on pain etiology. In some aspects, the patient can be determined to be responsive or non-responsive based on one or more biomarkers. In some aspects, the stimulation routine can be provided only to patients that are determined to be responsive to a stimulation routine. For example, in some aspects, a stimulation routine can be prescribed only to patients determined to be responsive.

A system can comprise a plurality of electrodes and a stimulator in communication with each electrode of the plurality of electrodes. The stimulator can be configured to generate a transdermal stimulation routine between at least two electrodes of the plurality of electrodes.

In some aspects, the stimulator can be battery powered. It is contemplated that implantable stimulators use batteries, but require long life to the detriment of the effectiveness of the stimulation routine. The stimulator of the disclosed apparatus, being operated transdermally and without need for a long life, can use more consistent batteries (e.g., lithium ion batteries or nickel cadmium batteries) or use AC current from a power cable, thereby applying a more consistent and more effective stimulation routine.

Also disclosed herein is a method of treating pain (e.g., chronic lower back). A plurality of electrodes can be positioned proximate to a spinal column. The spinal column can be stimulated transdermally by the electrodes in accordance with a therapeutic routine. In some aspects, positioning the plurality of electrodes can comprise positioning the plurality of electrodes anywhere along the spine between the spinous processes (e.g., midline between the T10 and L1 spinous processes). In further aspects, the therapeutic routine can comprise a biphasic rectangular waveform with 1 ms width pulses filled with a carrier frequency (e.g., a 5-10 kHz carrier frequency).

In some aspects, the disclosed systems and methods can be used for treating acute and chronic musculoskeletal pain (e.g. neck pain, bone fractures, etc.). In some aspects, the disclosed systems and methods can be used for treating post-surgical pain. In some aspects, the disclosed systems and methods can be used for treating peripheral neuropathy (e.g. diabetic neuropathy). In some aspects, the disclosed systems and methods can be used for treating cardiac pain. In some aspects, the disclosed systems and methods can be used for treating cancer- and/or chemotherapy-induced pain. In some aspects, the disclosed systems and methods can be used for treating acute visceral pain (kidney infections, etc.). In some aspects, the disclosed systems and methods can be used for treating pain associated with childbirth. Optionally, it is contemplated that the disclosed methods can be used to treat pain originating from stimulus, as contrasted with phantom limb pain. Thus, it is contemplated that the location of treatment along the spine can be adjusted depending upon the type of pain to be treated.

It is contemplated that efficacy of the treatment can decrease over time if the routine is maintained constant. To avoid this decrease over time, in some aspects, stimulation parameters can be varied. For example, in some aspects, one more stimulation parameters can be randomized. In other aspects, one or more stimulation parameters can be varied by predetermined levels. For example, in some aspects, the carrier frequency can be varied. In other aspects, the repetition frequency can be varied. In other aspects, the pulse width can be varied.

Further, it is contemplated that the disclosed method for applying a transdermal stimulation routine to the spine can be combined with additional treatment. For example, physical therapy (PT) is marginally effective for improving pain such as lower back pain. This is in part due to volitional effort being insufficient to overcome motor inhibitions that result from nociceptive (pain afferent) signaling. Accordingly, tSCS for pain treatment as disclosed herein can prime the nervous system to make PT more effective at retraining maladaptive movement patterns. This can address the root/cause of ongoing symptoms. Accordingly, in some aspects, stimulation alone can be primarily focused on symptom management, whereas stimulation combined with PT can be curative. For example, in some aspects, physical therapy can be performed during the transdermal stimulation routine to the spine. In further aspects, physical therapy can be performed after receipt of transdermal stimulation routine to the spine, while the patient is still experiencing reduced pain from the transdermal stimulation routine (e.g., within minutes, hours, days, or weeks of receiving the transdermal stimulation routine). Physical therapy can include strengthening and/or stretching muscles associated with the spine.

In some aspects, tSCS treatment can be ceased (no further sessions are performed) after a threshold reduction in pain. For example, tSCS treatment can be ceased after a perceived decrease in pain of 20%, or 30%, or 40%, or 50%, or 60%, or 70%, or 80%, or 90%, or more (relative to a starting pain level). In some aspects, tSCS treatment can be ceased (no further sessions are performed) after a reduction in pain in response to one session is not perceived. In some aspects, tSCS treatment can be ceased (no further sessions are performed) after a reduction in pain is not perceived for a threshold number of consecutive sessions (e.g., 2 sessions, 3 sessions, 4 sessions, or more). That is, once treatment does not provide any additional marginal improvement, treatment can be ceased.

### Exemplary Process

In an exemplary non-liming process, an electrode (cathode) can be placed at each of T8, T10, and L1 vertebrae if the focus of the stimulation routine is treating low back pain, or anywhere along the spine for other pain treatment types (e.g. T6 for cardiac pain), depending on the spinal cord innervation level of the target organ of treatment. Cervical stimulation can be used in conjunction with other more localized electrodes (e.g. T10/L1 for low back pain, T6 for cardiac pain, etc.), to potentiate activation of the endogenous descending pain inhibitory circuitry located in the brainstem region which controls its output. Anodes (ground electrodes) can be placed bilaterally over the iliac crests and/or paraumbilically (over the abdomen) and/or over the clavicles. Each electrode can be independently controlled on a respective channel. Stimulation can be turned on at each channel/electrode with a set carrier frequency (e.g., 10 kHz). This frequency can make stimulation tolerable at high intensities necessary to reach the spinal cord). This frequency can be referred to as a carrier frequency.

A stimulation routine can be provided by each electrode. The stimulation routine can comprise a plurality of repetitions of pulses provided at the carrier frequency. In some aspects, for each electrode, the stimulation routine can be set at a maximum intensity that the patient can endure. For each electrode, the intensity can initially be raised to a baseline level at which the patient feels the stimulation. This can be recorded as a sensation threshold. This data can be collected to understand the variability of sensation perception across patients. In addition, establishing a sensation threshold can be used to define the lower limit of the therapeutic ranges of stimulation. The intensity can then be increased to a patient tolerance threshold. For example, the tolerance threshold can be the intensity causing an amount of discomfort that a patient can endure for a full therapeutic session (e.g., 20 minutes, or about 20 minutes, or 20-30 minutes). The tolerance threshold (and, optionally, the sensation threshold) can be obtained for each electrode. Once the tolerance threshold is obtained for each electrode, each of the electrodes can be set at their tolerance thresholds for simultaneous delivery of therapeutic stimulation. The stimulation routine can be delivered for a full therapeutic session. In some aspects, the intensity can be sustained at the tolerance thresholds for the full therapeutic session. In other aspects, the intensities can be intermittently reduced below the tolerance thresholds. For example, the intensity can be cyclically maintained at the tolerance threshold for a first time period (e.g., about 1 minute), followed by an off period or a lower intensity period of less duration (e.g. 30 seconds). This can, for example, provide temporary relief to the patient and potentially provide the means to avoid/reduce the development of stimulation tolerance which may reduce the efficacy of treatment.

Referring to FIG. 16, in some optional aspects, an input device 30 can be in communication with the computing device 1001. A user (e.g., a patient or a clinician) can use the input device 30 to control at least one parameter of the stimulation routine. In some optional aspects, the user can interface with the computing device through a display device (e.g., monitor), which optionally can be part of or associated with the input device 30. The computing device can provide a portal via the display device through which the user can interface with the computing device.

In some aspects, the stimulation routine can be controlled automatically by the computing device 1001 based on the feedback received from the patient (e.g., through a patient interface). For example, the user can use the input device to provide input as to whether the intensity is too high. The computing device can be configured to lower the intensity in response to receiving user input that the intensity is too high. In further aspects, the user can use the input device to provide input as to whether the intensity is too low (e.g., a higher intensity can be tolerated). The computing device can be configured to increase the intensity in response to receiving user input that the intensity is too low.

In exemplary aspects, the stimulation routine can be provided in sessions. In some exemplary, optional aspects, the sessions can last from 15 minutes to 2 hours (e.g., about 20 minutes to about 30 minutes). Sessions can be spaced out by one or more days. For example, sessions can be provided at a frequency of no more than 4 times per week, or no more than 3 times per week, or no more than twice per week, or no more than once per week. Accordingly, it is contemplated that therapeutic results can be achieved with intermittent treatment sessions. In other aspects, treatment sessions can be provided daily or semi-daily. A treatment can be provided over a span of sessions (e.g., 4-30 sessions, or 6-20 sessions, or 10-15 sessions). It is contemplated that lasting therapeutic benefits (pain reduction) can continue to be experienced after cessation of treatment. In some aspects, treatment can be provided at the discretion of the patient. For example, the patient can use the system at his/her discretion, when pain relief is needed and for as long as needed. In some aspects, the system can be an in-home system.

### Exemplary Machine Learning System

As discussed herein, a predictive model may be generated by a computing device using machine learning techniques and algorithms. The predictive model may be used to determine whether a pain metric is high or low (e.g., the same as, or reduced from, a baseline of status quo pain level). The predictive model may be a result of applying one or more machine learning models and/or algorithms to sample data associated with a plurality of pain metrics. Machine learning is a subfield of computer science that gives computers the ability to learn without being explicitly programmed. Machine learning platforms include, but are not limited to, naïve Bayes classifiers, support vector machines, decision trees, neural networks, and the like.

For example, a computing device may be used to receive and analyze sample data associated with a plurality of pain metrics using one or more machine learning models and/or algorithms. The sample data may include one or more pain metrics. In some aspects, the pain metrics can include a number of blood biomarker data sets associated with one or more patients experiencing various levels of pain. In further or alternative aspects, the pain metrics can include EEG data associated with one or more patients experiencing various levels of pain. The sample data may include a first portion (“first sample data”) and a second portion (“second sample data”). The second sample data may include pain metrics that are labeled as either being high (e.g., the same as or greater than a previous level for the patient) or low (reduced from a previous level for the patient). The computing device may utilize the one or more machine learning models and/or algorithms to determine which of the one or more features of the sample data are most closely associated with high pain metrics versus low pain metrics (or vice-versa). Using those closely associated features, the computing device may generate a predictive model. The predictive model (e.g., a machine learning classifier) may be generated to classify a pain metric as being high or being low based on analyzing the pain metrics.

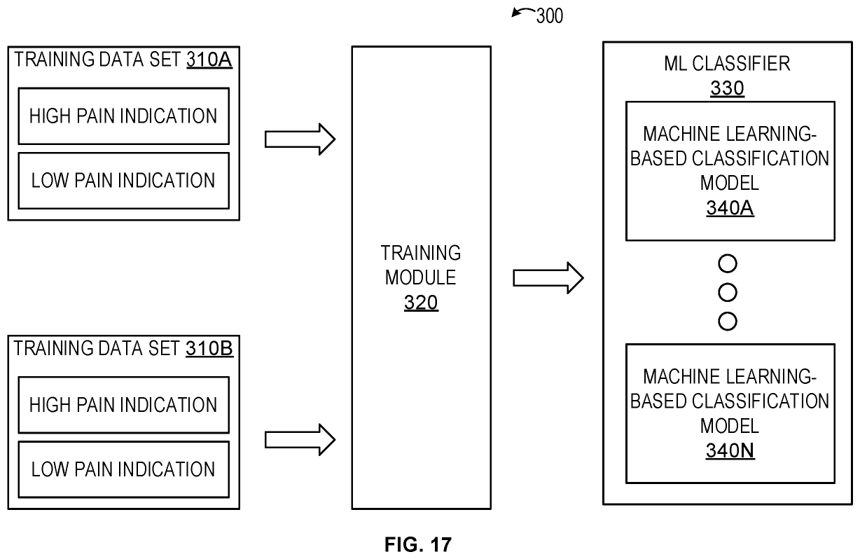

Turning now to FIG. 17, a system 300 is shown. The system 300 may be configured to use machine learning techniques to train, based on an analysis of one or more training data sets 310A-310B by a training module 320, at least one machine learning-based classifier 330 that is configured to classify a pain metric (e.g., a biomarker or data from an EEG scan) as being high or low. The training data set 310A (e.g., a first portion of the second sample data) may comprise biomarkers or EEG data from the same patient or from a plurality of patients. The training data set 310B (e.g., a second portion of the second sample data) may also comprise biomarkers or EEG data from the same patient or from a plurality of patients. The training data can include labels such as high or low pain indications.

A second portion of the second sample data may be randomly assigned to the training data set 310B or to a testing data set. In some implementations, the assignment of data to a training data set or a testing data set may not be completely random. In this case, one or more criteria may be used during the assignment, such as ensuring that similar numbers of material samples with different labels are in each of the training and testing data sets. In general, any suitable method may be used to assign the data to the training or testing data sets, while ensuring that the distributions of high or low pain indication labels are somewhat similar in the training data set and the testing data set.

The training module 320 may train the machine learning-based classifier 330 by extracting a feature set from the first portion of the second sample data in the training data set 310A according to one or more feature selection techniques. The training module 320 may further define the feature set obtained from the training data set 310A by applying one or more feature selection techniques to the second portion of the second sample data in the training data set 310B that includes statistically significant features of positive examples (e.g., associated with low pain indications) and statistically significant features of negative examples (e.g., associated with high pain indications).

The training module 320 may extract a feature set from the training data set 310A and/or the training data set 310B in a variety of ways. The training module 320 may perform feature extraction multiple times, each time using a different feature-extraction technique. In an embodiment, the feature sets generated using the different techniques may each be used to generate different machine learning-based classification models 340. For example, the feature set with the lowest pain indications may be selected for use in training. The training module 320 may use the feature set(s) to build one or more machine learning-based classification models 340A-340N that are configured to indicate whether or not pain metrics are associated with a low pain indication or a high pain indication.

The training data set 310A and/or the training data set 310B may be analyzed to determine any dependencies, associations, and/or correlations between extracted features and the high pain indication/low pain indication labels in the training data set 310A and/or the training data set 310B. The identified correlations may have the form of a list of features that are associated with different high pain indication/low pain indication labels. The features may be considered as variables in the machine learning context. The term “feature,” as used herein, may refer to any characteristic of an item of data that may be used to determine whether the item of data falls within one or more specific categories. By way of example, the features described herein may comprise one or more pain metric result attributes. The one or more pain metric result attributes may include a change in EEG measurement (e.g. shift in patterns of brain activity, measured using either the whole brain spectral power density or localized to specific brain regions of interest, such as those known to process pain related sensory information), a change in blood biomarker level, a combination thereof, or the like.

A feature selection technique may comprise one or more feature selection rules. The one or more feature selection rules may comprise a pain metric result attribute occurrence rule. The pain metric result attribute occurrence rule may comprise determining which pain metric result attributes in the training data set 310A occur over a threshold number of times and identifying those pain metric result attribute that satisfy the threshold as candidate features. For example, any pain metric result attributes that appear greater than or equal to 3 times in the training data set 310A may be considered as candidate features. Any pain metric result attributes appearing less than 3 times may be excluded from consideration as a feature. Any threshold amount may be used as needed.

A single feature selection rule may be applied to select features or multiple feature selection rules may be applied to select features. The feature selection rules may be applied in a cascading fashion, with the feature selection rules being applied in a specific order and applied to the results of the previous rule. For example, the pain metric result attribute occurrence rule may be applied to the training data set 310A to generate a first list of pain metric result attributes. A final list of candidate features may be analyzed according to additional feature selection techniques to determine one or more candidate groups (e.g., groups of pain metric attributes that may be used to predict whether a pain metric result of a biomarker or EEG data comprises high pain indication or low pain indication). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., high pain indication vs. low pain indication).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature groups. Forward feature selection is an iterative method that begins with no features in the machine learning model. In each iteration, the feature which best improves the model is added until an addition of a new feature does not improve the performance of the machine learning model. In an embodiment, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 320 has generated a feature set(s), the training module 320 may generate a machine learning-based classification model 340A based on the feature set(s). A machine learning-based classification model may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

The training module 320 may use the feature sets extracted from the training data set 310A and/or the training data set 310B to build a machine learning-based classification model 340A-340N for each classification category (e.g., sufficient quality, insufficient quality). In some examples, the machine learning-based classification models 340A-340N may be combined into a single machine learning-based classification model 340. Similarly, the machine learning-based classifier 330 may represent a single classifier containing a single or a plurality of machine learning-based classification models 340 and/or multiple classifiers containing a single or a plurality of machine learning-based classification models 340.

The extracted features (e.g., one or more pain metric result attributes) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 330 may comprise a decision rule or a mapping for each candidate pain metric result attribute to assign a pain metric result to a class (e.g., sufficient quality vs. insufficient quality).

The candidate pain metric result attributes and the machine learning-based classifier 330 may be used to predict a label (e.g., high pain indication vs. low pain indication) for pain metric results in the testing data set (e.g., in the second portion of the second sample data). In one example, the prediction for each pain metric result in the testing data set includes a confidence level that corresponds to a likelihood or a probability that the corresponding pain metric result belongs in the predicted high pain indication/low pain indication status. The confidence level may be a value between zero and one, and it may represent a likelihood that the corresponding pain metric result belongs to a high pain indication/low pain indication status. In one example, when there are two statuses (e.g., high pain indication and low pain indication), the confidence level may correspond to a value p, which refers to a likelihood that a particular pain metric result belongs to the first status (e.g., high pain indication). In this case, the value 1−p may refer to a likelihood that the particular pain metric result belongs to the second status (e.g., low pain indication). In general, multiple confidence levels may be provided for each pain metric result and for each candidate pain metric result attribute when there are more than two statuses. A top performing candidate pain metric result attribute may be determined by comparing the result obtained for each pain metric result with the known high pain indication/low pain indication status for each pain metric result in the testing data set (e.g., by comparing the result obtained for each pain metric result with the labeled high pain indications). In general, the top performing candidate pain metric result attribute will have results that closely match the known sufficient quality/insufficient quality statuses.

The top performing pain metric result attribute may be used to predict the high pain indication/low pain indication quality status of a pain metric result of pain metric. For example, new sample data associated with a pain metric result of a new material sample may be determined/received. The pain metric data may be provided to the machine learning-based classifier 330 which may, based on the top performing candidate pain metric result attribute, classify the pain metric result of the pain metric as indicating high pain or low pain.

### EXAMPLES

The following sections discuss exemplary, non-limiting uses of the disclosed systems and methods for purposes of reducing pain in patients.

### Example 1

### Outcomes

The initial therapeutic efficacy of tSCS intervention (12-21 sessions of 30 minutes of tSCS 3 times/week for 4-7 weeks) in 10 subjects with cLBP can be shown. For example, individuals with cLBP can report significantly reduced pain and disability outcome following 12-21 sessions of tSCS.

The effects of tSCS on objective sensorimotor outcomes, including patient-specific movement biomechanics, muscle activation patterns, spinal cord excitability and brain structural and functional connectivity at baseline and after tSCS can be determined. For example, patients can have improved biomechanics and increased paraspinal muscle activation during functional tasks, increased spinal cord excitability and normalized brain fMRI patterns following 12-21 sessions of tSCS as compared to baseline.

### Methods:

16 patients are used (randomized into tSCS group (n=8) or sham stimulation (n=8) with non-specific low-back pain (NS-LBP). Patients are recruited from the research database and/or the VA clinic. Upon arrival patients undergo a battery of assessments, comprising patient reported outcomes: Visual Analogue Scale (current pain scores) and current opioid use status (Yes/No, mg/day if yes). Patients then are asked to perform at least 3 trials of 3 repetition of sit-to-stand (STS). Patient biomechanics are recorded using Kinect system as previously described. In addition, surface (EMG) from paraspinal muscles at T10 and L5, rectus femoris and medial gastrocnemius (electrodes placed bilaterally) are recorded during STS. Patients undergo a 20-minute fMRI brain scan to assess resting state functional connectivity of the Default Mode Network (DMN) and Insula-Cingulate as well as lumbar spine scan.

Transcutaneous spinal cord stimulation therapy: Patients are asked to come to UCSF laboratory (3 times a week for 4 weeks) where they can be administered 20-30 minutes of stimulation. pre- and post-VAS scores can be collected after each session. For tSCS, two round stimulating electrodes can be placed midline between T10 and L1 spinous processes, as cathodes and 2 rectangular pads can be placed symmetrically on the skin over the iliac crests as anodes. The 5-channel stimulator generates pain-free biphasic rectangular waveform with 1 ms width pulses filled with 5-10 kHz carrier frequency. Stimulation frequency can be set at 15-30 Hz for both channels, while stimulation intensity are administered intermittently (1 min on at higher intensity with 30 second off/or at lower intensity) for the duration of 20-30 minutes. Following 12 sessions of stimulation, patients can undergo the same battery of sensorimotor assessments as described for baseline.

Neurophysiological assessment. Transcutaneous spinal cord stimulating electrodes can be placed at T10 and L1 as described above. Participants can be lying in a supine position on the exam table during the assessment and can be asked to stay relaxed. The stimulation can be delivered as single, 1-ms monophasic square-wave pulses every 6 s. The stimulation intensity can be increased in 2 mA increments from 2 to 100 mA or the maximum tolerable intensity. A minimum of three stimuli can be delivered at each intensity. Recruitment curves of the 8 muscles can be collected for the T10 and L1 stimulation locations. Magnitudes of the spinally evoked motor potentials (MEPs) can be calculated by measuring the area under the curve within a time window which can be manually selected for each muscle. The onset of the time window can be defined from the overlaid responses based on the earliest inclination from the baseline across all stimulation intensities. Magnitudes can be plotted as a function of stimulation intensity to determine the recruitment curve for each muscle at each location of spinal stimulation. Paraspinal muscle quality outcomes can be collected from the cLBP patients on a separate visit using magnetic resonance imaging (MRI) of the lumbar spine and fat fractionation of the paraspinal muscles for each spinal segment can be calculated as described previously.

Data analysis: The primary outcome measures including MEP amplitude (mV) and latency (ms) and can be measured using electromyography (EMG, Delsys) from 8 lower limb muscles bilaterally; muscle sEMG root mean square (mV), pattern of activation (RMS ratios between muscles), pain scores can be assessed using Visual Analogue Scale (0-10 scores), Oswestry Disability Index (0-100). Maximum sagittal vertical axis (degrees), maximum and minimum L5S1, hip, knee and ankle joint angles (degrees) maximum and minimum pelvis, torso, thigh and shank velocity (m/s) and accelerations (m/s²) can be measured using Motion Capture System. Resting state functional connectivity between key brain regions (Fz) involved in pain processing can be measured using fMRI. Data-driven, unsupervised machine learning approach, non-linear principal component analysis can be used to assess variable interaction within the multidimensional data. The stable, retained principal components can be analyzed using ANOVA and Tukey post hoc.

### Example 2

This example 1) establishes the initial efficacy of 12 tSCS therapy sessions to improve pain and objective sensorimotor outcome in 16 patients with cLBP and 2) shows determined neurological signatures of responders (cLBP improvement) and non-responders (no pain relief) to tSCS individuals. The product of this study is the first ever noninvasive spinal cord stimulation therapy to treat cLBP. This example lays down a foundation for the further development of a safe and effective therapy for pain such as cLBP, which can lead to a mass market production of the device and its rapid patient availability.

### I. Executive Summary:

Unmet medical need: As a leading cause of disability worldwide, cLBP represents a significant medical and socioeconomic problem with estimated health care spending of $87 billion/annually. Tightly intertwined with cLBP pandemic, is the ongoing opioid-use crisis, as opioid prescription remains first line of treatment for cLBP. Safe, non-addictive therapies that are ideally non-invasive are needed.

The initial discovery/observation: Transcutaneous spinal cord stimulation is a promising non-invasive neuromodulation alternative to epidural SCS in the field of spinal cord injury (SCI) that to date has not been tested for treatment of cLBP. The results are encouraging, as over 70% of patients with intractable pain had over 50% pain relief after 1 year of treatment. To date, SCS for treatment of cLBP has only been delivered via epidural electrodes on the spinal cord dorsal surface, requiring neurosurgical implantation. Although the implantable electrical stimulators have a low rate of adverse events compared to the opioid-related incidence of death, secondary complications associated with surgical intervention still occur. Given that epidural and transcutaneous SCS activate similar neuronal networks, the use of tSCS for treatment of pain in cLBP has advantages due to its non-invasive nature.

Specific Focus: In the first step of this example, the feasibility of 4 weeks of tSCS (3 times/week for a total of 12 sessions) to improve patient reported outcomes (e.g. pain and disability scores) was determined as well as objective measures of sensorimotor function, including sit-to-stand biomechanics, surface electromyography (sEMG). This enables prospectively examining the mechanisms of tSCS effects on patients' sensorimotor function. In the second step of this example, neurological substrates/neural signatures responsible for pain relief vs pain persistence in patients with cLBP can be identified. In the third step, the EEG activation patterns during tSCS to those previously identified in response to invasive SCS in prior EEG studies can be compared.

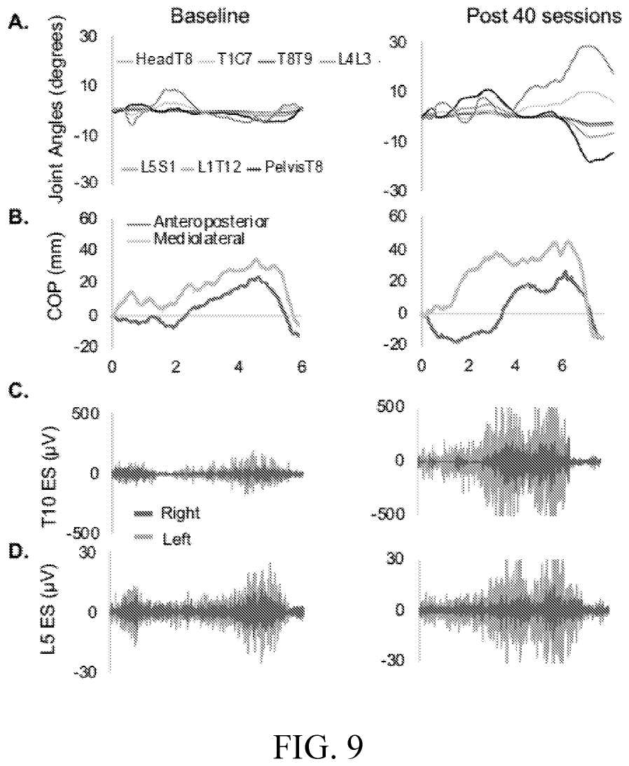

Product: (FIG. 9): This product can be the first ever noninvasive spinal cord stimulation therapy to treat cLBP. This work lays down a foundation for the further development of a safe and effective therapy for cLBP, which can lead to a mass market production of the device and its rapid patient availability. Mechanistically focused study design of this work can advance understanding of how different biopsychosocial factors contribute to cLBP which can enable a more targeted treatment approach to improve patient outcomes.

### II. Details of Medical Need and Competitive Landscape

According to Global Burden of Disease, low back pain is one of the leading causes of disability worldwide. The lifetime prevalence of cLBP is estimated to be 84% with 55% of patients predicted to have 10 low back pain episodes over their lifespan. Although multiple guidelines advocate for sparing use of opioids in the conservative management of LBP, opioids are still prescribed for treatment of acute and chronic pain conditions. Previously, an observational trial, reported that nearly 20% of the eligible patients with LBP received long-term opioids during the treatment process.

Opioids modulate their potent analgesic effects acutely by inducing pre- and postsynaptic Ca++ currents as well as K+ inward currents within the nociceptive afferents, attenuating their excitability effectively reducing the release of pronociceptive/proinflammatory neuropeptides. However, both chronic pain and chronic opioid use promote neuroinflammation in the limbic brain structures resulting in negative emotional states, contributing to the propensity of opioid misuse in patients with chronic pain. On the contrary, SCS is a relatively safe and effective therapy for cLBP that has the potential to replace opioids to manage chronic pain. Importantly, in one clinical trial, electrical SCS was shown to reduce mean daily use of opioids in patients and over 60% of individuals either reduced or excluded opioids at the last follow-up. Despite this success, the long-term efficacy of SCS needs further improvement, and objective comprehensive sensorimotor evaluations of patients are required for SCS evolution. The advantages of this system are based on the empirical efficacy of invasive SCS, with further advancement towards non-invasive technology. To characterize potential limitations of tSCS efficacy (e.g. invasive SCS efficacy rates are 50-60%) neurological signatures of tSCS therapy in responders (cLBP improvement) vs. non-responders (no pain relief) can be profiled and identified, building on and extending the successful investigation of EEG-based pain biomarkers. This can maximize resources such that the outcomes of further studies can rapidly expand the current understanding of neurophysiological mechanisms underlying SCS which can be beneficial to ensure further device development and a much higher success rate of tSCS therapy going forward.

### III. Discovery and Product Opportunity

Continued rigorous research on neuromodulation in SCI field has led to a development of a transcutaneous spinal cord stimulation device that potentiates and activates similar neural structures as epidural stimulation, but non-invasively, requiring only the placement of stimulating electrode(s) on the skin over the lumbosacral spinal cord. Numerous studies in SCI have demonstrated that tSCS potentiates lumbosacral spinal cord excitability enabling motor functions, (e.g. independent standing, postural control) in patients with chronic complete motor paralysis. Disclosed herein is a novel application of transcutaneous spinal cord stimulation for treatment of chronic low back pain. Although exemplary stimulation devices are known, these stimulators have not yet been used or tested for treatment of chronic low back pain conditions.

### IV. Catalyst Specific Exemplary Study

Step 1: Determine the effects of 12 sessions of tSCS on patient-reported outcomes and objective sensorimotor outcomes. 15 patients with non-specific low-back pain (NS-LBP) are used.

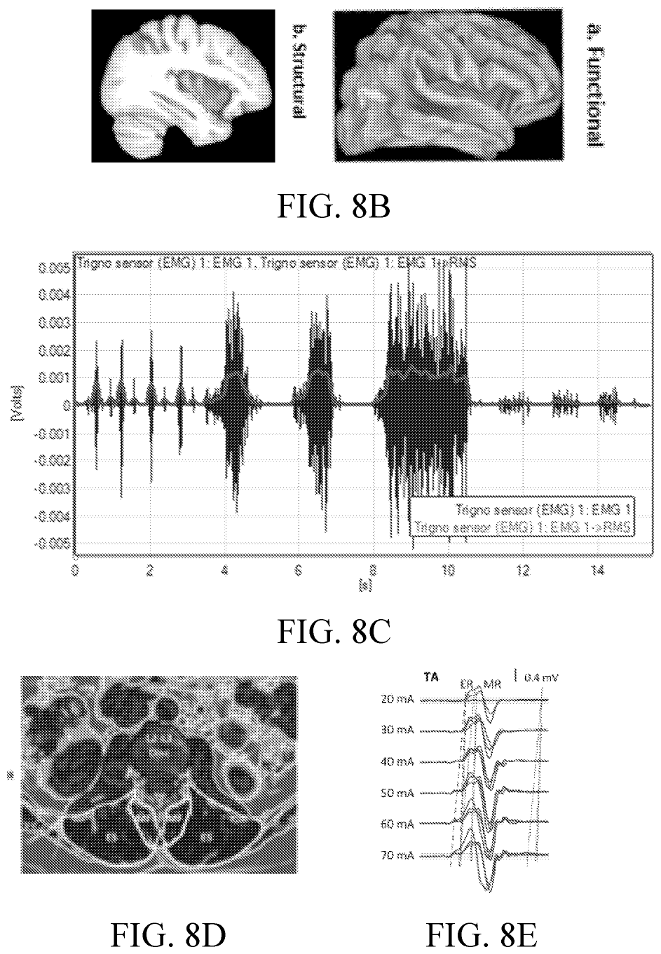

Patients can be recruited from the research database and/or the VA clinic. Upon arrival patients undergo a battery of assessments, comprising patient reported outcomes: Visual Analogue Scale (current pain scores) and current opioid use status (Yes/No, mg/day if yes). Patients are then asked to perform at least 3 trials of 3 repetition of sit-to-stand (STS). Patient biomechanics are recorded using Kinect system as previously described. In addition, surface (EMG) from paraspinal muscles at T10 and L5, rectus femoris and medial gastrocnemius (electrodes placed bilaterally) are recorded during STS. Paraspinal muscle quality outcomes are collected from the cLBP patients on a separate visit using magnetic resonance imaging (MRI) of the lumbar spine and fat fractionation of the paraspinal muscles for each spinal segment can be calculated as described previously (FIG. 8). Transcutaneous spinal cord stimulation therapy: Patients are asked to come to UCSF laboratory (3 times a week for 4 weeks) where they are administered 20-30 minutes of stimulation. Pre- and post-VAS scores can be collected after each session. For tSCS, two-three round stimulating electrodes are placed midline between C5-C6, T10 and L1 spinous processes, as cathodes and 2 rectangular pads are placed symmetrically on the skin over the iliac crests as anodes. The 5-channel stimulator generates pain-free biphasic rectangular waveform with 1 ms width pulses filled with 5-10 kHz carrier frequency. Stimulation frequency is set at 15-30 Hz for both channels, while stimulation intensity can be administered intermittently (1 min on at higher intensity with 30 second off/or at lower intensity) for the duration of 20-30 minutes.

Step 2: Identify neurological signatures of “pain relief” vs “pain persistence” derived from brain fMRI and EEG assessments are compared between baseline vs post 12 tSCS sessions. EEG are recorded using a 64-channel, high-frequency active electrode EEG headcap during stimulation optimization for pain relief. Neurological changes associated with tSCS are also assessed using brain fMRI scan to capture resting state functional connectivity of the Default Mode Network (DMN) and Insula—Cingulate before and after 20 minutes of tSCS. Structural brain MRI sequences are performed to assess cortical thickness and other gray matter volumes (FIG. 8). These assessments are done at baseline (prior to therapy) and post 12 sessions. Using EEG source localization algorithms, the active brain regions during tSCS to the active regions pre and post tSCS measured using fMRI are compared.

Step 3: Compare the EEG outcomes from a prior set of cLBP patients treated with epidural (implanted) SCS to the EEG outcomes from the patient cohort in the current study treated with non-invasive tSCS. Data analysis: The primary outcome measures including electromyography amplitude (mV) and latency (ms) and can be measured using electromyography (EMG, Delsys) from trunk and lower limb muscles bilaterally; muscle sEMG root mean square (mV), pattern of activation (RMS ratios between muscles), pain scores can be assessed using Visual Analogue Scale (0-10 scores), Oswestry Disability Index (0-100). Maximum sagittal vertical axis (degrees), maximum and minimum L5S1, hip, knee and ankle joint angles (degrees) maximum and minimum pelvis, torso, thigh and shank velocity (m/s) and accelerations (m/s²) can be measured using Motion Capture System or Kinect. Resting state functional connectivity between key brain regions (Fz) involved in pain processing can be measured using fMRI. One can use data-driven, unsupervised machine learning approach, non-linear principal component analysis to assess variable interaction within the multidimensional data. The stable, retained principal components can be analyzed using ANOVA and Tukey post hoc.

### Example 3

### Background

Chronic neuropathic pain (NP) affects approximately 53% of patients with spinal cord injury (SCI) with 1/3 of individuals experiencing pain as severe and debilitating. Neuropathic pain following SCI is categorized in relation to individual's neurologic level of injury as above-level, at-level, and below-level pain. Patients often describe NP as stabbing, sharp-electrical, shooting, or burning pain that can occur spontaneously in various regions of the body. Over 50% of individuals with SCI experience severe at-level pain defined as pain localized within one dermatome rostral and three dermatomes caudal to the injury and is often concomitant with mechanical and thermal hypersensitivity (e.g., allodynia and hyperalgesia). Approximately 35% of patients experience below-level pain within 5 years after SCI with similar NP characteristics. Below-level pain is most frequently experienced in the lower extremities with a more gradual onset and lower rates of dysesthesia and allodynia than at-level pain. The symptoms can be evoked, often by otherwise non-painful stimuli, as well as occur spontaneously. Any manifestation of NP further impairs the patient's quality of life adding to the challenges of the SCI-induced sensorimotor paralysis resulting in overall poor physical, cognitive, social and emotional well-being.

A number of pharmacological interventions for NP exist. However, pain is often refractory to pharmacological management and is associated with unwanted side effects (e.g. constipation or toxicity and increased risk of addiction or abuse). There is inconclusive evidence about the efficacy of non-pharmacological options, such as exercise, transcutaneous electrical nerve stimulation (TENS), and psychological or behavioral therapies (e.g. cognitive behavioral therapy) for SCI-related NP. A recent meta-analysis on medically refractory NP (not limited to SCI), has found consistent evidence in support of neuromodulation-based interventions (e.g. spinal cord stimulation, deep brain stimulation, etc.) for treatment of NP. However, the authors highlight the fact that the meta-analysis suffers from a low quality of the included studies. Another systematic review has likewise concluded that more vigorous clinical studies investigating the potential for SCS for treatment of NP after SCI are warranted given the high propensity of NP amongst individuals with SCI and its deleterious consequences on patients' quality of life.

The efficacy of dorsal column electrical stimulation to inhibit pain was first described over 50 years ago. Since then, several large clinical trials have investigated electrical spinal cord stimulation (SCS) as an alternative therapy for treatment and management of pain. The results are encouraging, as over 70% of patients with intractable pain had over 50% pain relief after 1 year of treatment. To date, SCS for treatment of chronic pain has been delivered via epidural electrodes on the spinal cord dorsal surface, requiring neurosurgical implantation. Although, the implantable electrical stimulators have a low rate of adverse events, compared to the opioid-related incidence of death, secondary complications associated with surgical intervention still occur.

In recent years, experimental application of epidural SCS has gained substantial interested in the SCI community. A number of studies have demonstrated the efficacy of epidural lumbosacral spinal cord stimulation to enable voluntary movement, standing and stepping after years of SCI-induced paralysis. Continued rigorous research of neuromodulation in SCI field has led to a development of a transcutaneous spinal cord stimulation device that potentiates and activates similar neural structures as epidural stimulation, but non-invasively, requiring only the placement of stimulating electrode(s) on the skin over the lumbosacral spinal cord. One of the key innovative features is the use of a specific pulse configuration with a carrier frequency of 5-10 kHz that minimizes discomfort by suppressing the sensitivity of cutaneous nociceptors when used at energies required to transcutaneously reach the spinal networks. Proof-of-concept studies have clearly demonstrated that non-invasive tSCS is able to reach and activate spinal cord networks. Gerasimenko et al., showed that tSCS at vertebral level T11 can induce involuntary stepping-like movements in non-injured humans when their legs are placed in a gravity-neutral position. Simultaneous independent stimulation at the C5, T11, and L1 vertebrae induced coordinated stepping-like movements with greater amplitude compared to stimulation at T11 alone. Thus, tSCS is an established neurophysiological tool that, depending on the location of stimulation, uncovers synergistic multi-segmental convergence of descending and ascending, most likely propriospinal networks on the lumbosacral neuronal circuitries associated with locomotor activity. Furthermore, similar to epidural SCS, studies using tSCS have demonstrated significant motor function improvements, enabling and/or improving trunk control, standing and stepping in individuals with SCI. Overall, recent neurophysiological studies suggest that tSCS and the epidural SCS applied to lumbosacral enlargements activated common neuronal structures with identical spinal evoked EMG responses.

### Neurophysiological Mechanisms of SCS Effects

The SCS-induced recovery of motor function following SCS, in general, is thought to occur due to counteraction of the loss of the tonic supraspinal drive by the exogenous electrical stimulation which raises the central state of excitability, enabling reactivation of the neural structures that were otherwise dormant in the persistent state of immobility due to paralysis. Surprisingly, given the initial rise of SCS to treat pain, very few of the existing neuromodulation focused studies in SCI have assessed efficacy of SCS for treatment of neuropathic pain following SCI, with opioids still considered for management of neuropathic pain Opioids have a potent analgesic effects acutely by inducing pre- and postsynaptic Ca⁺⁺ currents as well as K⁺ inward currents within the nociceptive afferents which attenuating their excitability effectively reducing the release of pronociceptive/proinflammatory neuropeptides. However, both chronic pain and chronic opioid use promote neuroinflammation in the limbic brain structures resulting in negative emotional states, contributing to the propensity of opioid misuse in patients with chronic pain. On the contrary, SCS has been demonstrated as a safe and effective therapy for chronic pain that has the potential to replace opioids to manage pain conditions. Although the exact mechanisms of the powerful pain inhibitory effects of SCS are still under investigation, several modes of actions have been elucidated though basic animal studies. Excessive nociceptive inputs cause wind up of the wide dynamic range (WDR) neurons which project to the lateral spinothalamic pathway ultimately triggering abnormal signaling in higher brain structures responsible for the perception of pain. Spinal cord stimulation increases γ-amino-butyric acid (GABA) release which reduces and shortens long-term potentiation, thereby modulating hyperexcitability of WDR neurons in the dorsal horn. In addition, SCS activates neurons in the rostroventral medulla (RVM) and the locus coeruleus in the brainstem facilitating descending inhibition of nociceptive signaling. Thus, SCS modulates/restores the endogenous pain inhibitory mechanisms at the spinal and supraspinal centers. Given the therapeutic supraspinal effects of SCS applied at the lumbosacral spinal cord, application of spinal cord stimulation at the cervical level, closer to the brainstem regions involved in the endogenous descending inhibition of pain, is an important therapeutic target of neuromodulation. This idea is supported by patient reports who experience significant low back pain relief while with cervical spinal cord stimulation. Moreover, SCS induces peripheral vasodilation through its antidromic activation of small diameter afferent fibers with the subsequent peripheral release of calcitonin gene related peptide (CGRP), a potent vasodilator, as well as through inhibition of efferent sympathetic outflow. Increased blood flow to the affected peripheral structures may improve tissue healing if neuropathic pain is accompanied with a peripheral injury.

Chronic pain is a neuroinflammatory disorder mediated by both the nervous and immune systems. Circulating immune cells such as neutrophils, monocytes, and T cells are recruited to sites of tissue damage, infiltrating the peripheral and central nervous systems. Upon activation these cells express a diverse profile of inflammatory mediators, such as cytokines/chemokines and proteases, affecting peripheral sensory or central second order neurons and/or other immune cells involved in nociception/pain regulation. Immune cells residing in the CNS (microglia and astrocytes) have a well-established role contributing to central sensitization and pain. Recently, Parisien et al., have identified thousands of dynamic transcriptional changes by performing a transcriptome-wide analysis in peripheral immune cells of individuals with acute LBP who were followed for 3 months. The authors found transient neutrophil-mediated up-regulation of inflammatory responses to be protective against development of chronic pain. This exemplary study did not only identify gene arrays as potential diagnostic biomarkers for acute to chronic pain transition but also provided important mechanistic insights underlying the chronification of pain. To date, only one case study has investigated the effects of neuromodulation (epidural spinal cord stimulation) on peripheral blood transcriptomics. The authors demonstrated significant downregulation of several key pro-inflammatory genes, such as members of the MAP kinase family, TLRs-1, 2, and 4, and LY96, which interacts with TLR4, following epidural spinal cord stimulation intervention. Reducing systematic inflammation, present in individuals with SCI may be another important therapeutic target, since systemic inflammation has been linked to maintenance of chronic pain states. Therefore, in this example 1) potential difference in the peripheral immune blood cell transcriptomics in the responders vs. non-responders to SCS can be identified, 2) specific changes in the transcriptomics in the responders as a mechanistic read out for the SCS-induced pain relief can be identified. The validated methodology to use global transcriptomic changes in the peripheral immune cells to have diagnostic and prognostic values in acute SCI can be implemented.

### Preliminary Studies

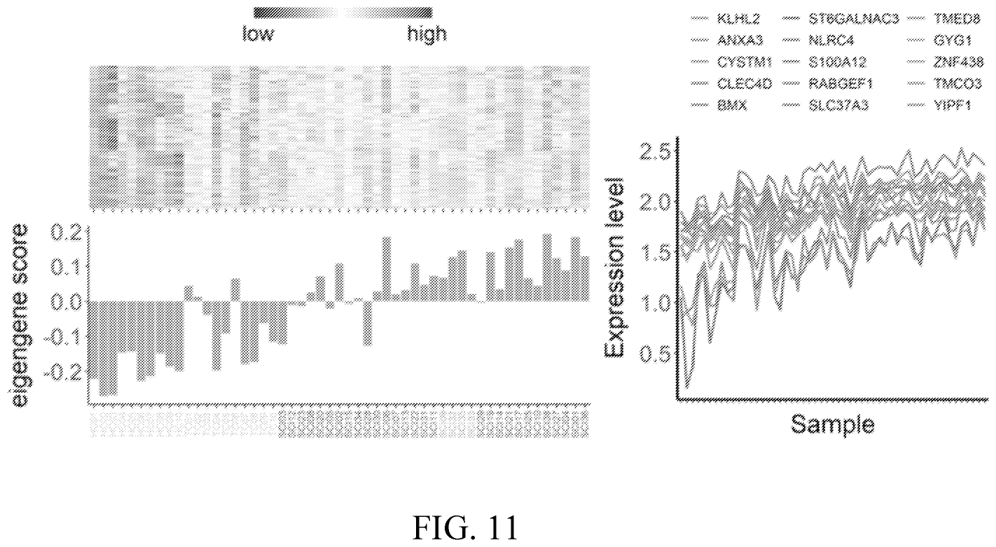

A first pilot clinical trial on safety and feasibility of transcutaneous spinal cord stimulation in children with SCI funded by National Center of Neuromodulation for Rehabilitation (NIH/NICHD) has been conducted. The first specific aim of that project was to determine proof-of-principle, feasibility and safety of scTS for acute potentiation of the upright sitting posture and trunk control in children (n=9) with SCI (FIG. 9). Lumbosacral tSCS is well-tolerated in children with SCI whose injury level is at least two segments above the placement of the stimulating electrodes (T11 and L1). Stimulation led to immediate significant increase in trunk extension and potentiated independent upright sitting posture in 7 tested children). The goal of the second aim (n=2) was to determine safety and feasibility of tSCS in combination with activity-based locomotor training (ABT) for potentiating upright posture and trunk control in children with SCI. Over a total of 63 scTS+AB-LT session in two participants, the cumulative exposure to scTS in combination with daily activity-based locomotor training (ABT) was safe and led to improvement in trunk control (FIG. 11).

### Limitation of Spinal Cord Stimulation and Potential Solutions

Presently, spinal cord stimulation (SCS) uses constant electrical stimulation without regard to ongoing pain state or unique pain related changes in the brain. SCS has four key drawbacks: 1) After implantation of SCS, effective stimulation parameters and electrode contacts must be chosen through a painstaking trial and error process by physician and technicians; 2) SCS often loses therapeutic effect after 1 year in up to 15% of patients; 3) The short battery life of most SCS devices has resulted in newer rechargeable batteries which are highly associated with treatment failure; 4) Before patients may use SCS, they must have an expensive one-week trial period because there has been no objective way to predict which patients receive pain relief.

To overcome these obstacles, brain-based biomarkers of high pain states in individual patients have been developed that have the potential to avert loss of effect, prolong battery life, assist in patient selection and automate choice of optimal stimulation parameters. Prior studies used averaging to identify common brain signatures across groups of patients, whereas recent studies have been critically focused on decoding pain biomarkers within an individual patient. Recent studies have collected data, a novel decoding scheme has been developed to predict stimulation parameters with very high accuracy. These personalized biomarkers are obtained using resting-state scalp recording with electroencephalography (EEG) and machine-learning tools that identify ‘neural signatures’ of chronic pain in individual patients. Neural signatures can comprise patient-specific mathematical models that incorporate power of brain oscillations and spatial coherence measures. With simultaneous EEG and SCS measurements during programming sessions, patterns of brain activity in response to effective therapeutic stimulation have been identified. Identifying which neural signatures, or neural biomarkers of pain are more responsive to spinal cord stimulation can aid in patient selection and avoid the need for a costly and painful trial period in the future. By recording EEG activity while varying SCS parameters, biomarkers that strongly predict pain or analgesia in individual patients with SCI-induced neuropathic pain can be identified.

### Feasibility of Obtaining EEG Recordings During Systematic SCS Parameter Testing

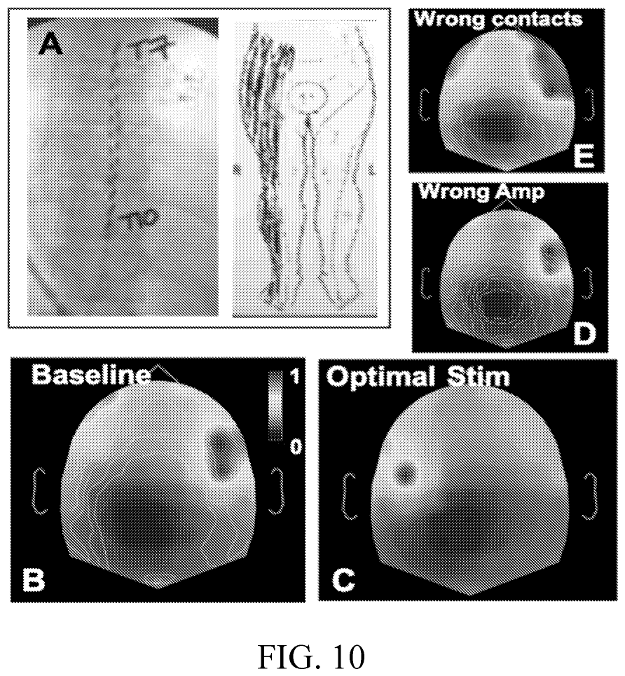

In a related study, high density EEG has been used to evaluate potential mechanism of action of cortical circuit activity on mediating the therapeutic efficacy of SCS (FIG. 10). Using an established recruitment pipeline, subjects with chronic low back and leg pain that are successfully being treated with SCS have been enrolled. Subjects have undergone 64 channel EEG recording while experiencing various stimulation programs. After a period of at least 24 hours when their stimulation is off (to allow for washout), stimulation programs can be systematically varied around their optimal configuration of stimulation parameters (correct contacts, amplitude, and frequency determined through conventional trial-and-error method). Starting at rest with stimulation off, EEG can be recorded for five minutes duration. Then, programs have been tested, including those where the programed contacts or amplitude differs from optimal settings.

Though one can successfully obtain reliable EEG recordings time locked to spinal stimulation, simple analyses such as focusing on a single frequency band (e.g. beta 12-30 Hz) over the entire cortex can be misleading. While signals can be ostensibly derived that may distinguish high pain states from effective SCS-induced low pain states, these patterns do not generalize across patients. Based on data, neural mechanisms of action of SCS are likely reflected in more nuanced metrics of network function.

### Blood Derived Biomarkers to Facilitate Mechanistic Understanding of Therapeutic Efficacy

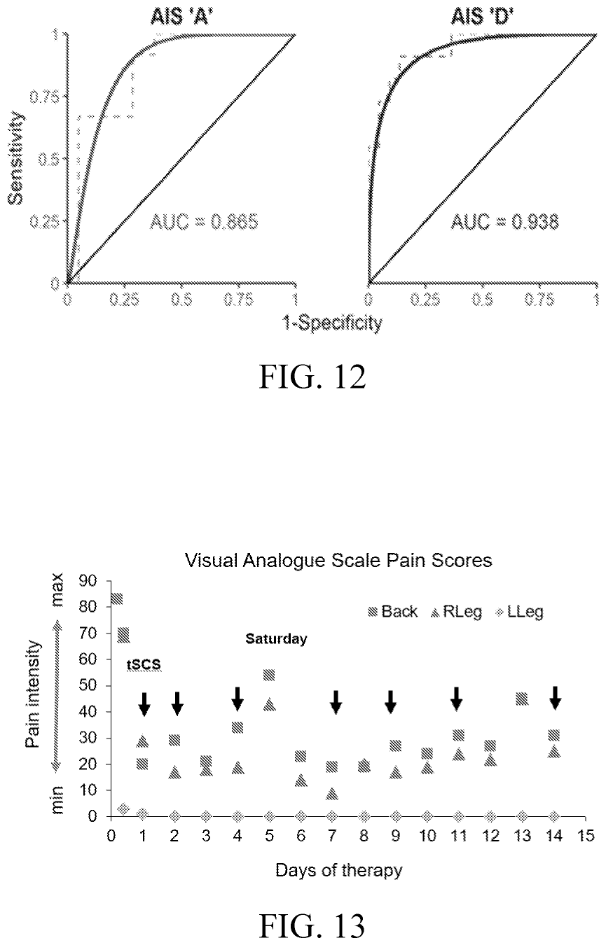

Despite the great potential of SCS, previous studies have shown that not all patients respond equally to the treatment. Given the time commitment required and the associated costs for SCS, classifying patients based on the probability of SCS success is of great importance. To this end, developing a biomarker signature for the stratification of patients into responders and non-responders to SCS can be a game changer leading to more personalized and precise decisions for each patient. Blood-based biomarkers for such decisions are already in mature stages of development in several other fields. For example, blood-based biomarkers are being used for predicting the response to antidepressants and immunotherapy for several types of cancer. The team in TRACK-SCI recently developed a novel pipeline and discovered white blood cell (WBC) transcriptomic signatures that can diagnose the initial severity of SCI with high accuracy. Specifically, gene co-expression network analysis has been used, and eigengene modules enriched in SCI patients compared to Healthy and Trauma Controls have been identified. FIG. 9 shows one of these modules (M13), which had the highest correlation to injury severity. To determine how well the expression patterns of these modules can collectively predict SCI severity, the SCI-enriched module eigengenes can be used as explanatory variables in a LASSO-regularized multinomial logistic regression model with AIS grade as the outcome. The model exhibited high accuracy of 72.7% in predicting the AIS grade with very impressive specificity and sensitivity for the AIS ‘A’ (AUC: 0.865) and AIS ‘D’ (AUC: 0.938) SCI patients (FIG. 12). This novel approach demonstrates that the transcriptomes of WBCs during the acute stage of SCI can predict injury severity and stratify patients into AIS grades. A similar approach can be utilized to identify eigengene modules behaving differently in SCI patients who respond to SCS versus non-responsive ones. A blood sample can be collected right before the SCS begins (baseline). Isolate and sequence RNA from WBCs can be isolated and sequenced, and the eigengene modules across all patients undergoing SCS can be generated. Then, eigengene modules whose expression is significantly different in SCI patients whose neuropathic pain was decreased by more than 50% (responders) compared to the rest (non-responders) can be identified. In addition, blood samples collected at the end of SCS sessions (transcutaneous and epidural) are completed can be used to discover 1) the molecular mechanisms altered in patients who responded positively to SCS and 2) differences in gene expression between patients who received transcutaneous versus epidural SCS.

### Objectives

This disclosure outlines the first dedicated neuromodulation study with the overall objective to determine efficacy of tSCS and eSCS for treatment of SCI-related neuropathic pain. Objective outcomes can be used, including neural and blood-based biomarkers that can be used in the futures as predictors of responders vs. non responders to SCS (>50% pain relief). Stimulation paradigms specific to treatment of chronic neuropathic pain have been identified, while establishing a feasible protocol for clinical implementation of SCS as a therapy for SCI NP. For example, tSCS can be a good first alternative to assess whether a patient might respond to SCS therapy for pain management before undergoing epidural implantation.

### Specific Aims/Hypotheses:

Aim 1a: Test efficacy of transcutaneous spinal cord stimulation therapy for treatment of neuropathic pain following SCI. 1b test the efficacy of epidural SCS therapy for treatment of NP after SCI.

Hypothesis 1: Epidural spinal cord stimulation leads to greater pain reduction (VAS/DN-4 scales) and higher quality of life improvement (WHOQOL-BREF) as compared to transcutaneous spinal cord stimulation in patients with chronic neuropathic pain

Aim 2: a) Identify neurological signatures of “pain relief” vs “pain persistence” derived from EEG assessments at baseline b) examine longitudinal changes in EEG patterns in responders vs. non-responders compared by tSCS and eSCS as a mechanistic read out for SCS-induced brain plasticity.

Hypothesis 2: a) Responders (>50% pain relief) and non-responders to therapy display distinct and EEG patterns b) SCS responders show greater top-down recruitment of descending pain inhibitory pathways from anterior cingulate (ACC) to insula or ACC to primary somatosensory cortex.

Aim 3: Perform exploratory analysis of blood-derived biomarkers that can predict responders (pain relief) vs. non-responders (pain persistence) to spinal cord stimulation.

Hypothesis 3: Blood-derived biomarkers (RNA-seq gene modules) derived from baseline (pre-therapy) blood draws differentiate/predict the responders vs. non-responders to spinal cord stimulation therapy.

### Study Design:

Aim 1. This is a randomized 2-arm interventional study with a cross-over design. This experimental design permits direct comparison between both types of spinal stimulation in the same subject. For this aim, 4 human subjects/per year over a 3-year period are recruited, with the goal of total 20 subjects (n=5/group). Inclusion criteria: Patients 6-month post incomplete SCI with NP in the trunk/lower extremities (DN4>4). Participants undergo baseline assessments, including the following patient reported outcomes (PROs): including pain intensity VAS, DN-4, and International SCI Pain Basic Data Set¹⁶, WHOQOL-BREF17 quality of life questionnaires, EEG and blood draws. The participants then receive 2 months of tSCS. The first arm (group A) receive 2 more months of tSCS followed by 1 month wash-out, and finally 2 months of eSCS. The second arm (group B) receive 4 months of epidural stimulation interleaved with 1-month wash-out. Assessments are repeated at the following time points: post 2 months of tSCS, post 1st wash out/pre-implant; post 2 months of eSCS (group B) or 4 months of tSCS (group A), post 2nd wash out, post eSCS (group A) and at follow up both groups; In addition, one can assess the difference in stimulation effect between the subgroups by quantifying changes in PROs, neurological and sensory outcomes during 2 months of tSCS in Group A and the first 2 months of epidural stimulation in Group B.

Transcutaneous spinal stimulation is delivered via skin surface electrodes placed over the spine during the intervention by using the transcutaneous stimulator, as previously published¹¹. Transcutaneous spinal cord stimulation optimization: On the first day of (after baseline assessments and before therapy) patients can undergo a stimulation optimization session to identify the individualized stimulation parameters that maximize pain relief. For transcutaneous spinal cord stimulation (tSCS) during this study, stimulating electrodes can be placed over the spine in the lower back and cervical regions, and the stimulation can be turned on at each location individually first, and then all two-three locations. The parameters, such as the number of stimulating electrodes, the intensity of stimulation at each as well as frequency can be changed in response to patient feedback to find the optimal pain-relieving parameters individualized for them. The patients can be asked to report any changes in their pain perception. The 5-channel stimulator generates pain-free biphasic rectangular waveform with 1 ms width pulses filled with 5-10 kHz carrier frequency. Patients can undergo post-treatment comprehensive assessments within 2 weeks of the last tSCS therapy session. Therapeutic application of transcutaneous spinal cord stimulation can then take place 3 times a week for 20-30 minutes in the laboratory settings using stimulation parameters identified during the stimulation optimization experiments. If there is no immediate identifiable pain relief with stimulation applied in the acute experiment, then the stimulation parameters used traditionally for epidural SCS can be approximated/adopted to the tSCS. The epidural stimulator can be surgically implanted in the operating room using established methods. The stimulation parameters can be adapted based on the functional and electrophysiological testing with most effective combination of parameters including electrode locations, pulse width and frequency.

Aim 2. In addition to patient reported outcomes, participants can undergo encephalography (EEG). The goal is to derive personalized EEG-based biomarkers of neuropathic pain in SCI, by obtaining resting-state scalp recording with EEG. In this aim, use machine learning tools such as support vector machine can be used to perform supervised classification of recorded neural data into responders vs non-responders. Neural biomarkers can comprise patient-specific mathematical models that incorporate power of brain oscillations and spatial coherence measures across EEG contacts. With simultaneous EEG and SCS measurements during programming sessions, patterns of brain activity in response to effective therapeutic stimulation can be identified, which informs software transcutaneous and epidural SCS. Identifying which neural signatures of neuropathic pain respond to spinal cord stimulation can aid in patient selection and may avoid the need for a costly and painful trial period in those patients who benefit most from the epidural SCS as determined in the study.

To characterize the dynamics of cortical network activity in relation to SCS therapy, one can combine EEG with machine learning methods to predict SCS responders and stimulation efficacy.

Overview/Rationale: To date, there are very limited studies evaluating the effect of SCS on neural activity as measured with EEG. Most studies using EEG focus on evoked potentials, seeking to characterize the role of SCS on brain responses to peripheral nerve stimulation. However, there is no clear understanding how endogenous EEG patterns in the resting state are affected by spinal cord stimulation. Further, many studies evaluate EEG during SCS in the operating room when pain reports are not available or fail to adequately control for off-target effects of SCS that are unrelated to pain. This study combines very common electrophysiological measurements with EEG in systematically controlled experiments to elucidate mechanisms of SCS efficacy that are distinct from dose-dependent or off-target effects. The ability to predict SCS trial success or mechanistically distinguish between electrical stimulation programs that differentially affect pain relief has significant potential to advance basic knowledge of chronic pain mechanisms. Finally, by characterizing an integrated mechanistic framework supporting SCS based pain relief, from RNA to EEG, an entirely new scope of scientific questions can be answered.

EEG Testing Protocol: EEG data collection can be performed across 3 visits per subject, each lasting at most 2 hours (see Study Timeline).

The first recording visit can take place before the patient receives their trial stimulator device, after they have received all necessary approvals for spinal cord stimulation therapy on the morning of their trial procedure. Patients can be brought to the EEG room and fitting with a 64 channel EEG headset sized to head circumference. EEG data can be sampled at 2048 Hz, using a 64+8 channel Biosemi ActiveTwo system (Biosemi Instrumentation), with a CMS-DRL reference. Four extra electrodes can be placed as follows: one on each mastoid (digitally linked, signal average used for subsequent re-referencing of the montage), one for EKG artifact, and another over the external battery to capture stimulation artifact. Standard physiological measurements can also be taken, such as EKG via leads placed on the patient's chest and galvanic skin response via 2 leads placed on 2 fingers from each hand. Five minute resting state EEG can be recorded while subjects sit comfortably with their eyes open and staring at a fixed point (an ‘X’ on blank computer screen) and again for 5 minutes with eyes closed.

The second EEG study visit occurs at the end of the first stimulation epoch (transcutaneous stimulation for all patients). 10 minutes of resting state EEG with the patient sitting can be recorded. The stimulator can be left on whatever settings were optimized for pain relief during the trial. The stimulator can then be turned off for at least 10 minutes and then subjects can be tested in 5 phases of 5 minutes each. There can be 5 minutes rest (washout) between each phase and subjects can report pain intensity NRS, VAS and MPQ as well as pain unpleasantness VAS during each phase. Subjects are blinded to the following stimulation conditions occurring in pseudorandom order (to account for order effects).
1. Subjects can be asked to sit comfortably for a period of 5 minutes. (Baseline)
2. After this, their spinal cord stimulation device can be turned on by the investigator using the remote control/clinician programmer using contacts that do not provide any pain relief, at the same amplitude and frequency as optimal settings. (′Wrong contact location)
3. Next, SCS can then be programmed to optimal settings except a frequency of 20 Hz (which has not been shown to be therapeutic)—(“Wrong frequency”)
4. The SCS can then be programmed to optimal settings except at 50% of optimal amplitude (“Wrong amplitude”)
5. Subjects can finally be tested again at optimal program settings.

The third EEG study visit occurs at the end of the second stimulation epoch (transcutaneous or epidural stimulation depending on randomized crossover). The fourth and final EEG study visit occurs at the end of the final stimulation epoch (epidural stimulation for all patients). Patients can be asked to turn off their stimulator for at least 12 hours prior to this visit to allow for therapeutic washout. EEG recordings can be collected again using the same 5 conditions as above.

EEG Data Preprocessing: For initial cleaning and processing of the raw datasets, an automated computational pipeline was developed and deployed using open-source EEG analysis packages implemented in the MATLAB computing environment. Raw datasets can be loaded into the pipeline and initially cleaned to produce adequate data for analysis. Baseline correction was first performed to remove DC offset effects and linear trends from the data. The data was then notch filtered at 60 Hz to remove electrical line noise from the ambient environment. Bad or defective electrode channels can be removed from the datasets and replaced by interpolating the surrounding usable channels. The first and last minute of the recording can be removed to isolate the typically most stable region of the data, then split into consecutive epochs of 1 second in duration. Artifacts generated by disturbances during the recording, such as pulses from the subject's heart or blinks, eye or jaw motion, can be identified using thresholding and Z-score analysis on select channels. Once these artifacts were identified, independent component analysis (ICA) was performed on the data to isolate individual statistically independent components, followed by time-locked coherence analysis to determine the components generated by the artifact signal. These artifactual components can then be removed to reduce the artifact signal from the original data stream. Finally, the dataset was re-referenced and labeled according to its effective stimulation condition.

EEG Spectral Analysis and Stimulation condition classification/modeling: The spectral power density of each epoch, averaged within canonical neural activity bands (delta, theta, alpha, beta, gamma) can be evaluated. To parameterize global network activity at each contact, a linear regression to the spectral power of each canonical frequency band can be fit, defining the spectral tilt value of each epoch as the slope of this curve. Next, using these calculations for each contact location as individual features and the set of values for each epoch as individual observations, each observation can be labeled based on the corresponding spinal cord stimulator program condition (e.g. baseline, optimal, wrong contact, etc.). A multi-class quadratic support vector machine (SVM) classification algorithm can then be applied to identify each trial and stimulator program condition. To improve the generalizability of the model and protect against overfitting the model to the training data, leave one out cross validation can be used on one second epochs across all patients. To reduce the influence of autocorrelations on overfitting, 5 epochs on either side of the test point from the training set can be removed.

EEG Source localization: After initial cleaning and processing of the raw data, clean datasets of recorded EEG data were obtained for each subject under distinct stimulation conditions. Bandpass filters were then applied to these cleaned datasets to isolate six different oscillatory bands in the neural activity signal (delta, theta, alpha, beta, low gamma, high gamma). For each dataset, dipole source localization was performed in software using the FieldTrip EEG analysis toolbox and final visualizations were prepared with the BrainNet Viewer tool, both implemented in MATLAB. First, mesh data representing the MNI-152 template brain was loaded from a BrainNet Viewer surface file. This mesh data was used to create a representative headmodel in the FieldTrip source localization pipeline, and dipole sources were fit to this coordinate space for each cleaned, filtered dataset. This process generated source localization results for each activity band across all subjects and stimulation conditions. These results were further processed in an automated software pipeline to iterate through each source to find and remove sources deemed to be subject-specific (source results unique to a single subject, present across all trial conditions). Then, sources held in common across subjects (source results present in the same trial condition across multiple subjects) were isolated and tallied. Results that were common across more than 50% of the subject population were highlighted for visualization and further analysis.

EEG spectral analysis with SVM classification can significantly distinguish responders from non-responders during the trial phase, lending insight to brain mechanisms of SCS based pain relief. Source localization studies on these data can help to clarify key brain circuits mediating such relief including the ACC and DLPFC. SVM on spectral tilt features can distinguish effective SCS programs (optimal) from ineffective (wrong contact, amplitude, etc.) programs. Further, EEG-SEG correlation, coherence and causality analyses are expected to show top-down flow of information, with spectral coherence being highest in frequency bands that are associated with key descending modulatory brain circuits. Finally, a composite analysis of RNA/gene profile clusters, SEG and EEG is expected to show significant correlations between factors that are independently associated with effective SCS induced pain relief. Possible latent variables identified with CCA may highlight important gene families that can guide future studies.

Aim 3: This study co-enrolls and gains new insights for patients in another DoD-funded clinical prospective study at the center (Transforming Research and Clinical Knowledge in SCI: TRACK-SCI). TRACK-SCI patients are scheduled for follow-up visits at 6 months post-injury, where motor, sensory, and pain status is evaluated. A blood sample is drawn as part of the TRACK-SCI biomarker discovery efforts in that follow-up visit. Specifically, total RNA is extracted from the white blood cells (WBCs), is sequenced, and transcriptomic signatures are identified through Weighted Gene Co-Expression Network Analysis (WGCNA). These signatures are then used for correlation with outcome measures. Since the TRACK-SCI blood samples are collected right before the disclosed intervention begins, specific transcriptomic signatures enriched in the SCI patients that respond to the stimulation can be identified. This can provide a vital resource and serve as a biomarker for identifying SCI patients more likely to respond to electrical stimulation therapy. A blood sample can also be collected at the end of the intervention to monitor the stimulation-induced, dynamic transcriptomic changes and their association with pain relief.

Sub aim 3.1. Use blood biomarker signatures to predict whether an SCI patient can respond positively (>50% pain reduction) to SCS.

In this sub aim, blood collected before the SCS sessions begin can be used to derive gene signatures and correlate their expression levels with the final outcome of the stimulation, which is whether SCI-induced chronic neuropathic pain was relieved. The SCI patients who report over 50% of pain relief can be the responders and the rest the non-responders. Specifically:

At least 6 months after their injury, SCI patients who experience neuropathic pain can donate a blood sample right before the first SCS session begins. The blood sample can be collected in an EDTA-coated tube and immediately centrifuged at 800 g for 15 minutes. The interphase layer that contains all WBCs can be carefully aspirated and transferred into a new tube containing 1× Red Blood Cell Lysis buffer. After a 15-minute incubation, the sample can be centrifuged at 800 g for 15 minutes. The supernatant can be removed, and the WBCs in the pellet can be dissolved in 1 mL of TRIZOL solution. Total RNA extraction using the classic TRIZOL protocol can follow. The RNA integrity can be assessed using the Agilent 2000 Bioanalyzer, and only samples with RIN scores above 7 can be sequenced. The RNA samples of high integrity can be submitted to the UC Davis Genome Core for RNAseq. The 3′-Tag RNAseq protocol that generates low-cost and low-noise gene expression profiling data can be used. Single-ended 50 base pairs at an estimated depth of 10 million reads per sample can be sequenced. Previous experience has shown that this depth is sufficient to detect most of the expressed genes.

After sequencing, the raw reads can be mapped against the human genome and derive the gene count matrix, which can be normalized for library size and composition bias. The normalized count matrix can then be used for differential gene expression (DGE) analysis between the two groups (responders vs. non-responders to SCS). Despite the usefulness of DGE, emerging evidence suggests that individual genes or molecules can be poor predictors of complicated biological and pathological systems; Multivariate predictors may therefore provide more robust biomarkers. To this end, gene co-expression network analysis can be applied to identify gene modules in the dataset that may represent reproducible biological processes with higher diagnostic value. After generating the eigengene modules across all the baseline samples (N=20), regularized logistic regression can be used to determine which modules (or a combination of them) can accurately predict whether an SCI patient will respond to SCS.

Sub aim 3.2. Use blood transcriptomics to discover novel molecular mechanisms involved in the relief/resolution of SCI-induced neuropathic pain.

Along with the baseline blood sample, each enrolled SCI patient can donate 3 additional blood samples at the end of each SCS session (see FIG. 10). The same procedures as in sub aim 3.1 can be followed, and normalized gene expression counts can be generated. That can two critical comparisons to be made:
1) The gene expression changes between responders and non-responders after each SCS session ends can be compared. This can be performed both on the single gene level through DGE analysis and via co-expression network analysis. These comparisons can allow us to decipher gene(s) and gene signatures altered in SCI patients who respond to SCS. That can be the first step toward unraveling potential molecular mechanisms governing neuropathic pain development and resolution and can provide therapeutic targets that do not require invasive methods such as SCS.
2) The second comparison that can be performed is the gene expression changes between patients who received transcutaneous and epidural SCS. Whether a different stimulation approach elicits different WBC transcriptomic reactions and whether those are associated with the outcome (pain relief) can be tested. This comparison can be very informative in a situation when one of the two stimulation methods is significantly more efficient than the other, as it can allow association of the response to SCS with specific genes and gene clusters.

### Statistical Plan and Data Analysis:

Power calculations on prior patient data (n=4) suggest that the factorial within-subjects cross-over design yields sufficient power (1−β>0.80) to detect significant main effects of tSCS (1−β=0.84) and some higher-order interactions, with moderate effect sizes expected in the patient sample (approximately η²>0.5). Given the cross-over within-subjects nature of the design, a sample size (n=6) is sufficient to show statistically significant results, and thus, providing proof-of-concept of the patient reported as well as physiological outcomes following SCS therapies.

Time and stimulation trial events can be treated as repeated measures and assessed along with the independent variables as a cross-over linear mixed model regression (LMM) using the Restricted Maximum Likelihood (REML) method. This statistical approach is robust to missing values and asymmetical group-sizes, violations of sphericity and other statistical features that are common realities in clinical studies but that can invalidate traditional linear models (e.g. ordinary least squares regression, analysis of variance). Based on the prior published work with transcutaneous stimulation using within-subjects LMM designs a power of 1-beta=0.8 for resolving p<0.05 with as few as n=5/group can be achieved. The use of multivariate and internal cross validation approaches can further boost power, enabling robust signal detection and maximal information gain while helping support rigorous and reproducible findings.

### Translational Potential

Since eSCS devices are FDA-approved and the tSCS device has already been IRB-approved for use in chronic low back pain subjects at UCSF, there is no need for IDE submission. However, the SCI field is currently in a state of uncertainty regarding stimulator use for neuropathic pain in SCI patients. It is not known if stimulators are as efficacious as they are in the able-bodied population. If one can identify neurological signatures that define responders/non-responders, one can target the optimum SCI patient subset with an effective non-pharmaceutical approach to pain management. The next step is to disseminate the knowledge gained by this study so that treating physicians can make evidence-based decisions and to validate the findings in a larger subset of SCI patients.

### Example 4

### Background

Chronic neuropathic pain (NP) affects approximately 53% of patients with spinal cord injury (SCI) with 1/3 of individuals experiencing pain as severe and debilitating. Neuropathic pain following SCI is categorized in relation to individual's neurologic level of injury as above-level, at-level, and below-level pain². Patients often describe NP as stabbing, sharp-electrical, shooting, or burning pain that can occur spontaneously in various regions of the body³. Over 50% of individuals with SCI experience severe at-level pain defined as pain localized within one dermatome rostral and three dermatomes caudal to the injury and is often concomitant with mechanical and thermal hypersensitivity (e.g., allodynia and hyperalgesia)⁴. Approximately 35% of patients experience below-level pain within 5 years after SCI with similar NP characteristics. Below-level pain is most frequently experienced in the lower extremities with a more gradual onset and lower rates of dysesthesia and allodynia than at-level pain⁵. The symptoms can be evoked, often by otherwise non-painful stimuli, as well as occur spontaneously. Any manifestation of NP further impairs the patient's quality of life adding to the challenges of the SCI-induced sensorimotor paralysis resulting in overall poor physical, cognitive, social and emotional well-being.

A number of pharmacological interventions for NP exist. However, pain is often refractory to pharmacological management⁷ and is associated with unwanted side effects (e.g. constipation or toxicity and increased risk of addiction or abuse). There is inconclusive evidence about the efficacy of non-pharmacological options, such as exercise, transcutaneous electrical nerve stimulation (TENS), and psychological or behavioral therapies (e.g. cognitive behavioral therapy) for SCI-related NP⁸. A recent meta-analysis on medically refractory NP (not limited to SCI), has found consistent evidence in support of neuromodulation-based interventions (e.g. spinal cord stimulation, deep brain stimulation, etc) for treatment of NP⁹. However, the authors highlight the fact that the meta-analysis suffers from a low quality of the included studies. Another systematic review has likewise concluded that more vigorous clinical studies investigating the potential for SCS for treatment of NP after SCI are warranted given the high propensity of NP amongst individuals with SCI and its deleterious consequences on patients' quality of life.

The efficacy of dorsal column electrical stimulation to inhibit pain was first described over 50 years ago. Since then, several large clinical trials have investigated electrical spinal cord stimulation (SCS) as an alternative therapy for treatment and management of pain. The results are encouraging, as over 70% of patients with intractable pain had over 50% pain relief after 1 year of treatment. To date, SCS for treatment of chronic pain has been delivered via epidural electrodes on the spinal cord dorsal surface, requiring neurosurgical implantation. Although, the implantable electrical stimulators have a low rate of adverse events, compared to the opioid-related incidence of death, secondary complications associated with surgical intervention still occur.

In recent years, experimental application of epidural SCS has gained substantial interested in the SCI community. A number of studies have demonstrated the efficacy of epidural lumbosacral spinal cord stimulation to enable voluntary movement, standing and stepping after years of SCI-induced paralysis. Continued rigorous research of neuromodulation in SCI field has led to a development of a transcutaneous spinal cord stimulation device that potentiates and activates similar neural structures as epidural stimulation, but non-invasively, requiring only the placement of stimulating electrode(s) on the skin over the lumbosacral spinal cord. One of the key innovative features is the use of a specific pulse configuration with a carrier frequency of 5-10 kHz that minimizes discomfort by suppressing the sensitivity of cutaneous nociceptors when used at energies required to transcutaneously reach the spinal networks. Proof-of-concept studies have clearly demonstrated that non-invasive tSCS is able to reach and activate spinal cord networks. Gerasimenko et al., showed that tSCS at vertebral level T11 can induce involuntary stepping-like movements in non-injured humans when their legs are placed in a gravity-neutral position. Simultaneous independent stimulation at the C5, T11, and L1 vertebrae induced coordinated stepping-like movements with greater amplitude compared to stimulation at T11 alone. Thus, tSCS is an established neurophysiological tool that, depending on the location of stimulation, uncovers synergistic multi-segmental convergence of descending and ascending, most likely propriospinal networks on the lumbosacral neuronal circuitries associated with locomotor activity. Furthermore, similar to epidural SCS, studies using tSCS have demonstrated significant motor function improvements, enabling and/or improving trunk control, standing and stepping in individuals with SCI. Overall, recent neurophysiological studies suggest that tSCS and the epidural SCS applied to lumbosacral enlargements activated common neuronal structures with identical spinal evoked EMG responses.

### Neurophysiological Mechanisms of SCS Effects

The SCS-induced recovery of motor function following SCS, in general, is thought to occur due to counteraction of the loss of the tonic supraspinal drive by the exogenous electrical stimulation which raises the central state of excitability, enabling reactivation of the neural structures that were otherwise dormant in the persistent state of immobility due to paralysis. Surprisingly, given the initial rise of SCS to treat pain, very few of the existing neuromodulation focused studies in SCI have assessed efficacy of SCS for treatment of neuropathic pain following SCI, with opioids still considered for management of neuropathic pain Opioids have a potent analgesic effects acutely by inducing pre- and postsynaptic Ca⁺⁺ currents as well as K⁺ inward currents within the nociceptive afferents which attenuating their excitability effectively reducing the release of pronociceptive/proinflammatory neuropeptides. However, both chronic pain and chronic opioid use promote neuroinflammation in the limbic brain structures resulting in negative emotional states, contributing to the propensity of opioid misuse in patients with chronic pain On the contrary, SCS has been demonstrated as a safe and effective therapy for chronic pain that has the potential to replace opioids to manage pain conditions. Although the exact mechanisms of the powerful pain inhibitory effects of SCS are still under investigation, several modes of actions have been elucidated though basic animal studies. Excessive nociceptive inputs cause wind up of the wide dynamic range (WDR) neurons which project to the lateral spinothalamic pathway ultimately triggering abnormal signaling in higher brain structures responsible for the perception of pain. Spinal cord stimulation increases γ-amino-butyric acid (GABA) release which reduces and shortens long-term potentiation, thereby modulating hyperexcitability of WDR neurons in the dorsal horn. In addition, SCS activates neurons in the rostroventral medulla (RVM) and the locus coeruleus in the brainstem facilitating descending inhibition of nociceptive signaling. Thus, SCS modulates/restores the endogenous pain inhibitory mechanisms at the spinal and supraspinal centers. Given the therapeutic supraspinal effects of SCS applied at the lumbosacral spinal cord, application of spinal cord stimulation at the cervical level, closer to the brainstem regions involved in the endogenous descending inhibition of pain, is an important therapeutic target of neuromodulation. This idea is supported by patient reports who experience significant low back pain relief while with cervical spinal cord stimulation. Moreover, SCS induces peripheral vasodilation through its antidromic activation of small diameter afferent fibers with the subsequent peripheral release of calcitonin gene related peptide (CGRP), a potent vasodilator, as well as through inhibition of efferent sympathetic outflow. Increased blood flow to the affected peripheral structures may improve tissue healing if neuropathic pain is accompanied with a peripheral injury.

Chronic pain is a neuroinflammatory disorder mediated by both the nervous and immune systems. Circulating immune cells such as neutrophils, monocytes, and T cells are recruited to sites of tissue damage, infiltrating the peripheral and central nervous systems. Upon activation these cells express a diverse profile of inflammatory mediators, such as cytokines/chemokines and proteases, affecting peripheral sensory or central second order neurons and/or other immune cells involved in nociception/pain regulation. Immune cells residing in the CNS (microglia and astrocytes) have a well-established role contributing to central sensitization and pain. Recently, Parisien et al., have identified thousands of dynamic transcriptional changes by performing a transcriptome-wide analysis in peripheral immune cells of individuals with acute LBP who were followed for 3 months. The authors found transient neutrophil-mediated up-regulation of inflammatory responses to be protective against development of chronic pain. This exemplary study did not only identify gene arrays as potential diagnostic biomarkers for acute to chronic pain transition but also provided important mechanistic insights underlying the chronification of pain. To date, only one case study has investigated the effects of neuromodulation (epidural spinal cord stimulation) on peripheral blood transcriptomics. The authors demonstrated significant downregulation of several key pro-inflammatory genes, such as members of the MAP kinase family, TLRs-1, 2, and 4, and LY96, which interacts with TLR4, following epidural spinal cord stimulation intervention. Reducing systematic inflammation, present in individuals with SCI may be another important therapeutic target, since systemic inflammation has been linked to maintenance of chronic pain states. Therefore, in this clinical trial, exploratory analysis can be performed to 1) identify any potential difference in the peripheral immune blood cell transcriptomics in the responders vs. non-responders to SCS, 2) identify specific changes in the transcriptomics in the responders as a mechanistic read out for the SCS-induced pain relief.

### Preliminary Studies

Previously, a first pilot clinical trial has been conducted on safety and feasibility of transcutaneous spinal cord stimulation in children with SCI funded by National Center of Neuromodulation for Rehabilitation (NIH/NICHD). The first specific aim of that project was to determine proof-of-principle, feasibility and safety of scTS for acute potentiation of the upright sitting posture and trunk control in children (n=9) with SCI (FIG. 9). It was established that lumbosacral tSCS is well-tolerated in children with SCI whose injury level is at least two segments above the placement of the stimulating electrodes (T11 and L1). Stimulation led to immediate significant increase in trunk extension and potentiated independent upright sitting posture in 7 tested children). The goal of the second aim (n=2) was to determine safety and feasibility of tSCS in combination with activity-based locomotor training (ABT) for potentiating upright posture and trunk control in children with SCI. Over a total of 63 scTS+AB-LT session in two participants, the cumulative exposure to scTS in combination with daily activity-based locomotor training (ABT) was safe and led to improvement in trunk control (FIG. 11).

### Limitation of Spinal Cord Stimulation and Potential Solutions

Presently, spinal cord stimulation (SCS) uses constant electrical stimulation without regard to ongoing pain state or unique pain related changes in the brain. SCS has four key drawbacks: 1) After implantation of SCS, effective stimulation parameters and electrode contacts must be chosen through a painstaking trial and error process by physician and technicians; 2) SCS often loses therapeutic effect after 1 year in up to 15% of patients; 3) The short battery life of most SCS devices has resulted in newer rechargeable batteries which are highly associated with treatment failure; 4) Before patients may use SCS, they must have an expensive one-week trial period because there is no objective way to predict which patients will receive pain relief.

To overcome these obstacles, brain-based biomarkers of high pain states in individual patients have been developed that have the potential to avert loss of effect, prolong battery life, assist in patient selection and automate choice of optimal stimulation parameters. Prior studies used averaging to identify common brain signatures across groups of patients, whereas recent studies have been critically focused on decoding pain biomarkers within an individual patient. Recent studies have collected data, a novel decoding scheme has been developed to predict stimulation parameters with very high accuracy. These personalized biomarkers are obtained using resting-state scalp recording with electroencephalography (EEG) and machine-learning tools that identify ‘neural signatures’ of chronic pain in individual patients. Neural signatures can comprise patient-specific mathematical models that incorporate power of brain oscillations and spatial coherence measures. With simultaneous EEG and SCS measurements during programming sessions, patterns of brain activity in response to effective therapeutic stimulation have been identified. Identifying which neural signatures, or neural biomarkers of pain are more responsive to spinal cord stimulation can aid in patient selection and avoid the need for a costly and painful trial period in the future. By recording EEG activity while SCS parameters are varied, biomarkers that strongly predict pain or analgesia in individual patients with SCI-induced neuropathic pain can be identified.

### Feasibility of Obtaining EEG Recordings During Systematic SCS Parameter Testing

In a related study, high density EEG has been used to evaluate potential mechanism of action of cortical circuit activity on mediating the therapeutic efficacy of SCS (FIG. 10). Using an established recruitment pipeline, subjects with chronic low back and leg pain that are successfully being treated with SCS have been enrolled. Subjects have undergone 64 channel EEG recording while experiencing various stimulation programs. After a period of at least 24 hours when their stimulation is off (to allow for washout), stimulation programs are systematically varied around their optimal configuration of stimulation parameters (correct contacts, amplitude, and frequency determined through conventional trial-and-error method). Starting at rest with stimulation off, EEG is recorded for five minutes duration. Then, programs including those where the programed contacts or amplitude differs from optimal settings have been tested.

Though one can successfully obtain reliable EEG recordings time locked to spinal stimulation, simple analyses such as focusing on a single frequency band (e.g. beta 12-30 Hz) over the entire cortex can be misleading. While signals can be ostensibly derived that may distinguish high pain states from effective SCS-induced low pain states, these patterns do not generalize across patients. Based on experience, neural mechanisms of action of SCS are likely reflected in more nuanced metrics of network function.

### Objectives

This proposal outlines the first dedicated neuromodulation study with the overall objective to determine efficacy of tSCS and eSCS for treatment of SCI-related neuropathic pain. Objective outcomes, including neural and blood-based biomarkers can be used in the futures as predictors of responders vs. non responders to SCS (>50% pain relief). Stimulation paradigms specific to treatment of chronic neuropathic pain have been identified, while establishing a feasible protocol for clinical implementation of SCS as a therapy for SCI NP. For example, tSCS can be a good first alternative to assess whether a patient might respond to SCS therapy for pain management before undergoing epidural implantation.

### Specific Aims/Hypotheses:

Aim 1a: Test efficacy of transcutaneous spinal cord stimulation therapy for treatment of neuropathic pain following SCI. 1b test the efficacy of epidural SCS therapy for treatment of NP after SCI.

Hypothesis 1: Epidural spinal cord stimulation lead to greater pain reduction (VAS/DN-4 scales) and higher quality of life improvement (WHOQOL-BREF) as compared to transcutaneous spinal cord stimulation in patients with chronic neuropathic pain

Aim 2: a) Identify neurological signatures of “pain relief” vs “pain persistence” derived from EEG assessments at baseline b) examine longitudinal changes in EEG patterns in responders vs. non-responders compared by tSCS and eSCS as a mechanistic read out for SCS-induced brain plasticity.

Hypothesis 2: a) Responder (>50% pain relief) and non-responders to therapy display distinct and EEG patterns b) SCS responders show greater top-down recruitment of descending pain inhibitory pathways from anterior cingulate (ACC) to insula or ACC to primary somatosensory cortex.

Aim 3: Perform exploratory analysis of blood-derived biomarkers that can predict responders (pain relief) vs. non-responders (pain persistence) to spinal cord stimulation.

Hypothesis 3: Blood-derived biomarkers (RNA-seq gene modules) derived from baseline (pre-therapy) blood draws differentiate/be able to predict the responders vs. non-responders to spinal cord stimulation therapy.

### Study Design:

Aim 1. This is a randomized 2-arm interventional study with a cross-over design. This experimental design permits direct comparison between both types of spinal stimulation in the same subject. For this aim, 4 human subjects/per year over a 3-year period can be recruited, with the goal of total 20 subjects (n=5/group). Inclusion criteria: Patients 6-month post incomplete SCI with NP in the trunk/lower extremities (DN4>4). Participants can undergo baseline assessments, including the following patient reported outcomes (PROs): including pain intensity VAS¹³, DN-4^{14,15}, and International SCI Pain Basic Data Set¹⁶, WHOQOL-BREF¹⁷ quality of life questionnaires, EEG and blood draws. The participants can then receive 2 months of tSCS. The first arm (group A) can receive 2 more months of tSCS followed by 1 month wash-out, and finally 2 months of eSCS. The second arm (group B) can receive 4 months of epidural stimulation interleaved with 1-month wash-out. Assessments can be repeated at the following time points: post 2 months of tSCS, post 1st wash out/pre-implant; post 2 months of eSCS (group B) or 4 months of tSCS (group A), post 2nd wash out, post eSCS (group A) and at follow up both groups; In addition, one can assess the difference in stimulation effect between the subgroups by quantifying changes in PROs, neurological and sensory outcomes during 2 months of tSCS in Group A and the first 2 months of epidural stimulation in Group B.

Transcutaneous spinal stimulation is delivered via skin surface electrodes placed over the spine during the intervention by using the transcutaneous stimulator, as previously published¹¹. Transcutaneous spinal cord stimulation optimization: On the first day of (after baseline assessments and before therapy) patients can undergo a stimulation optimization session to identify the individualized stimulation parameters that maximize pain relief. For transcutaneous spinal cord stimulation (tSCS) during this study, stimulating electrodes can be placed over the spine in the lower back and cervical regions, and the stimulation can be turned on at each location individually first, and then all two-three locations. The parameters, such as the number of stimulating electrodes, the intensity of stimulation at each as well as frequency can be changed in response to patient feedback to find the optimal pain-relieving parameters individualized for them. The patients can be asked to report any changes in their pain perception. The 5-channel stimulator generates pain-free biphasic rectangular waveform with 1 ms width pulses filled with 5-10 kHz carrier frequency. Patients can undergo post-treatment comprehensive assessments within 2 weeks of the last tSCS therapy session. Therapeutic application of transcutaneous spinal cord stimulation can then take place 3 times a week for 20-30 minutes in the laboratory settings using stimulation parameters identified during the stimulation optimization experiments. If there is no immediate identifiable pain relief with stimulation applied in the acute experiment, then the stimulation parameters used traditionally for epidural SCS can be approximated/adopted to the tSCS. The epidural stimulator can be surgically implanted in the operating room using established methods. The stimulation parameters can be adapted based on the functional and electrophysiological testing with most effective combination of parameters including electrode locations, pulse width and frequency.

Aim 2. In addition to patient reported outcomes, participants can undergo encephalography (EEG). The goal is to derive personalized EEG-based biomarkers of neuropathic pain in SCI, by obtaining resting-state scalp recording with EEG. Machine learning tools such as support vector machine can be used to perform supervised classification of recorded neural data into responders vs non-responders. Neural biomarkers can comprise patient-specific mathematical models that incorporate power of brain oscillations and spatial coherence measures across EEG contacts. With simultaneous EEG and SCS measurements during programming sessions, one can identify patterns of brain activity in response to effective therapeutic stimulation, which can inform future software development for the next-generation transcutaneous and epidural SCS. Identifying which neural signatures of neuropathic pain respond to spinal cord stimulation can aid in patient selection and may avoid the need for a costly and painful trial period in those patients who benefit most from the epidural SCS as determined in the study.

To characterize the dynamics of cortical network activity in relation to SCS therapy, one can combine EEG with machine learning methods to predict SCS responders and stimulation efficacy.

Overview/Rationale: To date, there are very limited studies evaluating the effect of SCS on neural activity as measured with EEG. Most studies using EEG focus on evoked potentials, seeking to characterize the role of SCS on brain responses to peripheral nerve stimulation. However, there is no clear understanding how endogenous EEG patterns in the resting state are affected by spinal cord stimulation. Further, many studies evaluate EEG during SCS in the operating room when pain reports are not available or fail to adequately control for off-target effects of SCS that are unrelated to pain. This study combines very common electrophysiological measurements with EEG in systematically controlled experiments to elucidate mechanisms of SCS efficacy that are distinct from dose-dependent or off-target effects. The ability to predict SCS trial success or mechanistically distinguish between electrical stimulation programs that differentially affect pain relief has significant potential to advance basic knowledge of chronic pain mechanisms. Finally, by characterizing an integrated mechanistic framework supporting SCS based pain relief, from RNA to EEG, an entirely new scope of scientific questions can be answered.

EEG Testing Protocol: EEG data collection can be performed across 3 visits per subject, each lasting at most 2 hours (see Study Timeline).

The first recording visit takes place before the patient receives their trial stimulator device, after they have received all necessary approvals for spinal cord stimulation therapy on the morning of their trial procedure. Patients can be brought to the EEG room and fitting with a 64 channel EEG headset sized to head circumference. EEG data can be sampled at 2048 Hz, using a 64+8 channel Biosemi ActiveTwo system (Biosemi Instrumentation), with a CMS-DRL reference. Four extra electrodes can be placed as follows: one on each mastoid (digitally linked, signal average used for subsequent re-referencing of the montage), one for EKG artifact, and another over the external battery to capture stimulation artifact. Standard physiological measurements can also be taken, such as EKG via leads placed on the patient's chest and galvanic skin response via 2 leads placed on 2 fingers from each hand. Five minute resting state EEG can be recorded while subjects sit comfortably with their eyes open and staring at a fixed point (an ‘X’ on blank computer screen) and again for 5 minutes with eyes closed.

The second EEG study visit occurs at the end of the first stimulation epoch (transcutaneous stimulation for all patients). One can record 10 minutes of resting state EEG with the patient sitting. The stimulator can be left on whatever settings were optimized for pain relief during the trial. The stimulator can then be turned off for at least 10 minutes and then subjects can be tested in 5 phases of 5 minutes each. There can be 5 minutes rest (washout) between each phase and subjects can report pain intensity NRS, VAS and MPQ as well as pain unpleasantness VAS during each phase. Subjects are blinded to the following stimulation conditions occurring in pseudorandom order (to account for order effects).
1. Subjects is asked to sit comfortably for a period of 5 minutes. (Baseline)
2. After this, their spinal cord stimulation device is turned on by the investigator using the remote control/clinician programmer using contacts that do not provide any pain relief, at the same amplitude and frequency as optimal settings. (′Wrong contact location)
3. Next, SCS is then programmed to optimal settings except a frequency of 20 Hz (which has not been shown to be therapeutic)—(“Wrong frequency”)
4. The SCS can then be programmed to optimal settings except at 50% of optimal amplitude (“Wrong amplitude”)
5. Subjects are finally tested again at optimal program settings.

The third EEG study visit occurs at the end of the second stimulation epoch (transcutaneous or epidural stimulation depending on randomized crossover). The fourth and final EEG study visit occurs at the end of the final stimulation epoch (epidural stimulation for all patients). One can ask patients to turn off their stimulator for at least 12 hours prior to this visit to allow for therapeutic washout. EEG recordings are collected again using the same 5 conditions as above.

EEG Data Preprocessing: For initial cleaning and processing of the raw datasets, an automated computational pipeline was developed and deployed using open-source EEG analysis packages implemented in the MATLAB computing environment. Raw datasets were loaded into the pipeline and initially cleaned to produce adequate data for analysis. Baseline correction was first performed to remove DC offset effects and linear trends from the data. The data was then notch filtered at 60 Hz to remove electrical line noise from the ambient environment. Bad or defective electrode channels were removed from the datasets and replaced by interpolating the surrounding usable channels. The first and last minute of the recording were removed to isolate the typically most stable region of the data, then split into consecutive epochs of 1 second in duration. Artifacts generated by disturbances during the recording, such as pulses from the subject's heart or blinks, eye or jaw motion, were identified using thresholding and Z-score analysis on select channels. Once these artifacts were identified, independent component analysis (ICA) was performed on the data to isolate individual statistically independent components, followed by time-locked coherence analysis to determine the components generated by the artifact signal. These artifactual components were then removed to reduce the artifact signal from the original data stream. Finally, the dataset was re-referenced and labeled according to its effective stimulation condition.

EEG Spectral Analysis and Stimulation condition classification/modeling: The spectral power density of each epoch can then be evaluated, averaged within canonical neural activity bands (delta, theta, alpha, beta, gamma). To parameterize global network activity at each contact, a linear regression can be fit to the spectral power of each canonical frequency band, defining the spectral tilt value of each epoch as the slope of this curve. Next, using these calculations for each contact location as individual features and the set of values for each epoch as individual observations, each observation can be labeled based on the corresponding spinal cord stimulator program condition (e.g. baseline, optimal, wrong contact, etc.). A multi-class quadratic support vector machine (SVM) classification algorithm can be applied to identify each trial and stimulator program condition. To improve the generalizability of the model and protect against overfitting the model to the training data, leave one out cross validation can be used on one second epochs across all patients. To reduce the influence of autocorrelations on overfitting, 5 epochs on either side of the test point from the training set can be removed.

EEG Source localization: After initial cleaning and processing of the raw data, clean datasets of recorded EEG data were obtained for each subject under distinct stimulation conditions. Bandpass filters were then applied to these cleaned datasets to isolate six different oscillatory bands in the neural activity signal (delta, theta, alpha, beta, low gamma, high gamma). For each dataset, dipole source localization was performed in software using the FieldTrip EEG analysis toolbox and final visualizations were prepared with the BrainNet Viewer tool, both implemented in MATLAB. First, mesh data representing the MNI-152 template brain was loaded from a BrainNet Viewer surface file. This mesh data was used to create a representative headmodel in the FieldTrip source localization pipeline, and dipole sources were fit to this coordinate space for each cleaned, filtered dataset. This process generated source localization results for each activity band across all subjects and stimulation conditions. These results were further processed in an automated software pipeline to iterate through each source to find and remove sources deemed to be subject-specific (source results unique to a single subject, present across all trial conditions). Then, sources held in common across subjects (source results present in the same trial condition across multiple subjects) were isolated and tallied. Results that were common across more than 50% of the subject population were highlighted for visualization and further analysis.

Outcomes: EEG spectral analysis with SVM classification can significantly distinguish responders from non-responders during the trial phase, lending insight to brain mechanisms of SCS based pain relief. Source localization studies on these data can help to clarify key brain circuits mediating such relief including the ACC and DLPFC. Extending preliminary data, one can expect that SVM on spectral tilt features can distinguish effective SCS programs (optimal) from ineffective (wrong contact, amplitude, etc.) programs. Further, EEG-SEG correlation, coherence and causality analyses are expected to show top-down flow of information, with spectral coherence being highest in frequency bands that are associated with key descending modulatory brain circuits. Finally, a composite analysis of RNA/gene profile clusters, SEG and EEG can show significant correlations between factors that are independently associated with effective SCS induced pain relief. Possible latent variables identified with CCA may highlight important gene families that can guide future studies.

Solutions to certain concerns: One potential concern is that testing of different stimulation program conditions rapidly for 5 minutes (with 5 minute washout time in between) may not allow enough time for any potential therapeutic benefit to take effect when switching to an effective program (“wash-in time”) or wear off if switching to an ineffective one (“wash-out time), which may confound interpretation. Though this problem was not observed during preliminary data collection (even with paresthesia free conditions), this can present a unique opportunity to study mechanisms of prolonged wash-in or wash-out in individual patients. Based on tracking simultaneously reported pain scores (VAS, NRS, MPQ), patients can be asked to wait until pain scores had either returned to pre-operative baseline or improved based on their personal experience during the trial phase.

It is possible that the combined SEG-EEG analysis may not show meaningful correlations or coherence values within comparable frequency bands. In this case, one can further analyze cross-frequency coupling measures which may reflect subthreshold periodic neural activity occurring in different anatomical geometries or tissue environments (e.g. due to low pass filtering of SEG from CSF between spinal cord and epidural space).

Aim 3: This study can co-enroll and gain new insights for patients in another DoD-funded clinical prospective study at the center (Transforming Research and Clinical Knowledge in SCI: TRACK-SCI). TRACK-SCI patients are scheduled for follow-up visits at 6 months post-injury, where motor, sensory, and pain status is evaluated. A blood sample is drawn as part of the TRACK-SCI biomarker discovery efforts in that follow-up visit. Specifically, total RNA is extracted from the white blood cells (WBCs), is sequenced, and transcriptomic signatures are identified through Weighted Gene Co-Expression Network Analysis (WGCNA). These signatures are then used for correlation with outcome measures. Since the TRACK-SCI blood samples are collected right before the disclosed intervention begins, specific transcriptomic signatures enriched in the SCI patients that respond to the stimulation can be identified. This can provide a vital resource and serve as a biomarker for identifying SCI patients more likely to respond to electrical stimulation therapy. A blood sample can also be collected at the end of the intervention to monitor the stimulation-induced, dynamic transcriptomic changes and their association with pain relief.

### Sub Aim 3.1. Use Blood Biomarker Signatures to Predict Whether an SCI Patient Responds Positively (>50% Pain Reduction) to SCS.

In this sub aim, blood collected before the SCS sessions begin can be used to derive gene signatures and correlate their expression levels with the final outcome of the stimulation, which is whether SCI-induced chronic neuropathic pain was relieved. The SCI patients who report over 50% of pain relief can be the responders and the rest the non-responders. Specifically:

At least 6 months after their injury, SCI patients who experience neuropathic pain can donate a blood sample right before the first SCS session begins. The blood sample can be collected in an EDTA-coated tube and immediately centrifuged at 800 g for 15 minutes. The interphase layer that contains all WBCs can be carefully aspirated and transferred into a new tube containing 1× Red Blood Cell Lysis buffer. After a 15-minute incubation, the sample can be centrifuged at 800 g for 15 minutes. The supernatant can be removed, and the WBCs in the pellet can be dissolved in 1 mL of TRIZOL solution. Total RNA extraction using the classic TRIZOL protocol can follow. The RNA integrity can be assessed using the Agilent 2000 Bioanalyzer, and only samples with RIN scores above 7 can be sequenced. The RNA samples of high integrity can be submitted to the UC Davis Genome Core for RNAseq. The 3′-Tag RNAseq protocol that generates low-cost and low-noise gene expression profiling data can be used. Single-ended 50 base pairs at an estimated depth of 10 million reads per sample can be sequenced. Previous experience has shown that this depth is sufficient to detect most of the expressed genes.

After sequencing, the raw reads can be mapped against the human genome and derive the gene count matrix, which can be normalized for library size and composition bias. The normalized count matrix can then be used for differential gene expression (DGE) analysis between the two groups (responders vs. non-responders to SCS). Despite the usefulness of DGE, emerging evidence suggests that individual genes or molecules can be poor predictors of complicated biological and pathological systems; Multivariate predictors may therefore provide more robust biomarkers. To this end, one can apply gene co-expression network analysis to identify gene modules in the dataset that may represent reproducible biological processes with higher diagnostic value. After generating the eigengene modules across all the baseline samples (N=20), one can use regularized logistic regression to determine which modules (or a combination of them) can accurately predict whether an SCI patient can respond to SCS.

### Sub Aim 3.2. Use Blood Transcriptomics to Discover Novel Molecular Mechanisms Involved in the Relief/Resolution of SCI-Induced Neuropathic Pain.

Along with the baseline blood sample, each enrolled SCI patient can donate 3 additional blood samples at the end of each SCS session (see FIG. 11). The same procedures as in sub aim 3.1 can be followed, and normalized gene expression counts can be generated. That can allow us to make two critical comparisons:
1) the gene expression changes between responders and non-responders after each SCS session ends can be compared. This can be done both on the single gene level through DGE analysis and via co-expression network analysis. These comparisons can allow deciphering gene(s) and gene signatures altered in SCI patients who respond to SCS. That can be the first step toward unraveling potential molecular mechanisms governing neuropathic pain development and resolution and can provide therapeutic targets that do not require invasive methods such as SCS.
2) The second comparison can be the gene expression changes between patients who received transcutaneous and epidural SCS. Whether a different stimulation approach elicits different WBC transcriptomic reactions and whether those are associated with the outcome (pain relief) can be tested. This comparison can be very informative in a situation when one of the two stimulation methods is significantly more efficient than the other, as it can allow association of the response to SCS with specific genes and gene clusters.

### Statistical Plan and Data Analysis:

Power calculations on prior patient data (n=4) suggest that the factorial within-subjects cross-over design yields sufficient power (1−β>0.80) to detect significant main effects of tSCS (1−β=. 84) and some higher-order interactions, with moderate effect sizes expected in the patient sample (approximately η²>0.5). Given the cross-over within-subjects nature of the design, a sample size (n=6) is sufficient to show statistically significant results, and thus, providing proof-of-concept of the patient reported as well as physiological outcomes following SCS therapies.

Time and stimulation trial events can be treated as repeated measures and assessed along with the independent variables as a cross-over linear mixed model regression (LMM) using the Restricted Maximum Likelihood (REML) method. This statistical approach is robust to missing values and asymmetical group-sizes, violations of sphericity and other statistical features that are common realities in clinical studies but that can invalidate traditional linear models (e.g. ordinary least squares regression, analysis of variance). Based on prior work with transcutaneous stimulation using within-subjects LMM designs, achieving a power of 1-beta=0.8 for resolving p<0.05 with as few as n=5/group is expected. The use of multivariate and internal cross validation approaches can further boost power, enabling robust signal detection and maximal information gain while helping support rigorous and reproducible findings.

### Access to Target Population/Enrollment Strategy

Potential subjects can be identified via Electronic Medical Record screening at ZSFG as well as from a pool of patients previously enrolled in the TRACK-SCI observational study. Approximately 25 patients per year have been enrolled in TRACK-SCI, giving a population of over 100 patients to offer enrollment. In the TRACK-SCI study, participants can be asked whether they wish to be contacted for future studies and those that agree can be contacted to ask whether they are interested in participating in this study. In order to give previous participants an opportunity to participate in this study, since neuropathic pain is a topic of great importance to many SCI patients, eligible participants enrolled prior to the addition of the future study contact question (where no previous indication against being contacted further was already noted by the study team) can be asked the question during routine TRACK-SCI follow-up conversations, or sent an email or letter where possible informing them of how to opt out of being contacted for future studies.

### Translational Potential

Since eSCS devices are FDA-approved and the tSCS device has already been IRB-approved for use in chronic low back pain subjects at UCSF, there is no need for IDE submission. However, the SCI field is currently in a state of uncertainty regarding stimulator use for neuropathic pain in SCI patients. It is not known if stimulators are as efficacious as they are in the able-bodied population. One can identify neurological signatures that define responders/non-responders and then target the optimum SCI patient subset with an effective non-pharmaceutical approach to pain management. The next step can be to disseminate the knowledge gained by this study so that treating physicians can make evidence-based decisions and to validate the findings in a larger subset of SCI patients.

### Example 5

This example tests a novel intervention; therefore, it has received the designation of a clinical trial. To begin testing the hypotheses outlined in the example, the team first had to onboard the study according to the IRB regulations as a clinical trial by obtaining registration at clinicaltrials.gov. In addition, the team had to obtain additional regulatory approvals at the Neuroscience Clinical Research Unit at the Sander Neuroscience Center at Mission Bay, which is the site for the conduction of all the patient assessments and administration of the experimental intervention. The regulatory steps included submission and approval of the study protocol by the NCRU directors/managers, obtaining access to key facilities within NCRU, such as MRI suite necessary for brain fMRI data collection, which required MRI safety training. The following pilot studies/equipment training were carried out as the regulatory documents were pending approval: electroencephalography (EEG) pilot data collection, fMRI protocol development/pilot studies, electromyography (EMG) equipment acquisition, pilot testing in combination with sit-to-stand biomechanics, RedCap project was created for collection of patient reported outcomes via surveys/questionnaires. All of the testing was necessary to train/prepare the team for efficient patient assessments/data collection.

Due to the limited number of staff dedicated to the project and limited avenues of patient recruitment, two patients have been tested with tSCS, who have undergone 12 sessions of tSCS therapy. Another patient has been consented and undergone baseline assessments, pending scheduling of their 12 therapy visits.

Aim 1: Determine the initial therapeutic efficacy of tSCS intervention (12 sessions of 20 minutes of tSCS 3 times/week for 4 weeks) in 10 subjects with cLBP.

Hypothesis 1: Individuals with cLBP report significantly reduced pain and disability outcome following 12 sessions of tSCS.

Aim 2: Determine the effects of tSCS on objective sensorimotor outcomes, including patient-specific movement biomechanics, muscle activation patterns, spinal cord excitability and brain structural and functional connectivity at baseline and after tSCS.

Hypothesis 2: Patients have improved biomechanics and increased paraspinal muscle activation during functional tasks, increased spinal cord excitability and normalized brain fMRI patterns following 12 sessions of tSCS as compared to baseline.

### Results

The first patient was a 72 year old male, injured in the bike accident 3.5 years ago, which resulted in the extruded disc in the lumbar region. Since then the patient has received conservative treatment including PT, acupuncture, coping with pain through meditation. The patient continued to have persistent pain in the lower back, radiating down the leg. His pain scores (Visual Analogue Scale) were 83 at enrollment, indicating severe pain. Just after 2 tSCS therapy sessions, the patient started reporting lower daily pain scores (30-40 on VAS scale), which remained at that lower level for the duration of the study and after its completion.

The second patient was a 56 year old female who over the years has received different diagnosis by different doctors including mild spondylosis, mild disc bulges, spinal stenosis of lumbar region with neurogenic claudication, L4-5 disc disease, straightening of the upper lumbar spine, sacral Tarlov cyst. The patient was managed with the conservative treatment including NSAIDS, opioids, injections, TENS unit. The patient reported having maximum VAS scores of 90 at enrollment, as the worst pain she experiences during the spikes, however, most of her daily persistent pain is around 50 on the VAS scale. This patient has also reported having shoulder pain at enrollment that impaired her range of motion, limiting several functional activities. Transcutaneous spinal cord stimulation did not alter this patient's pain experience in the lower back, as the patient continued to report LBP with VAS of 50 for the duration of the study. Interestingly, the patient has noticed significant improvements in her shoulder/neck pain levels and range of motion. She has regained some functional capacity of the right upper limb and has reported being able to use that arm to blow dry her hair, reach behind her back, which she has not been able to do due to pain in a long time. The participant has noticed this change since her initiation of therapy, and believes that the cervical spinal cord stimulation, which is one of the locations of tSCS administration in addition to the lumbosacral spinal cord stimulation, has led to that improvement.

EEG, fMRI, EMG and patient biomechanics data analysis enable better understanding of the underlying mechanisms of pain and individual response to tSCS (responder vs. non-responder phenotypes).

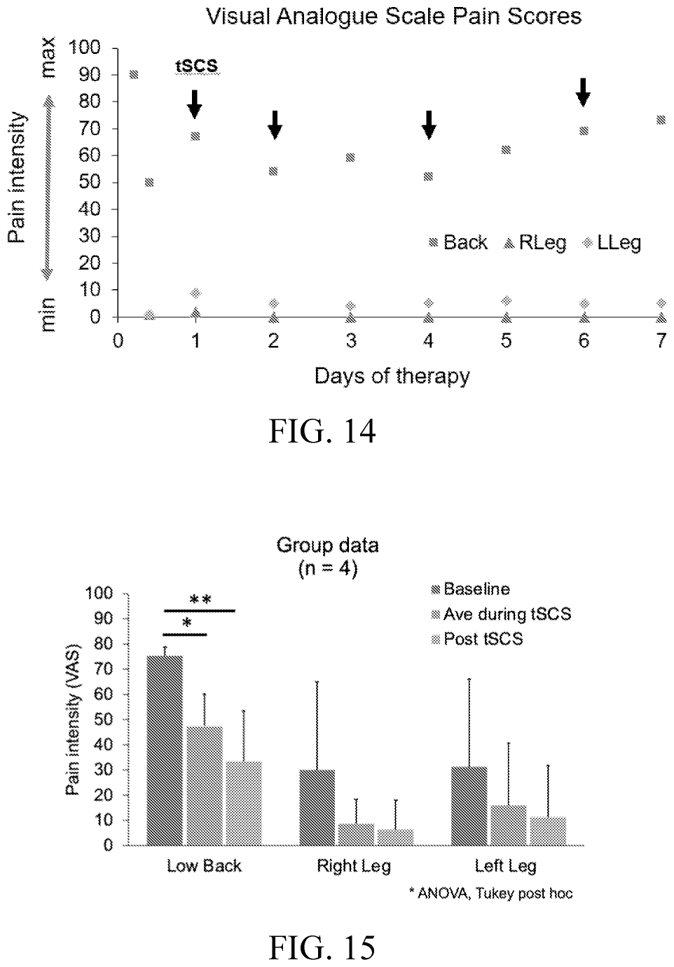

FIG. 13 shows a plot of pain after each session for the first patient. FIG. 14 shows a plot of pain after each session for the second patient.

### Example 6

FIG. 15 shows results of a study using 12 sessions, 3 sessions per week, 30 minutes per session. The data show 56% (42 points) reduction in back pain after tSCS therapy (p=0.005), 80% (24 points) reduction in right leg pain, and 64% (20 points) reduction in left leg pain.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method of treating pain in a patient, the method comprising:
applying a transdermal stimulation routine to a portion of a spinal cord to treat pain.

Aspect 2: The method of aspect 1, wherein the patient does not have a spinal cord injury that affects motor function.

Aspect 3: The method of aspect 2, wherein the patient does not have any spinal cord injury.

Aspect 4: A method of determining a transdermal stimulation routine for treating pain in a patient, the method comprising:
changing at least one of a pulse duration, a frequency or an amplitude of the transdermal stimulation routine based on a pain metric.

Aspect 5: The method of aspect 4, wherein the pain metric is an EEG measurement.

Aspect 6: The method of aspect 4, wherein the pain metric is a blood biomarker.

Aspect 7: The method of aspect 6, wherein the biomarker is RNA.

Aspect 8: The method of any one of aspects 4-7, wherein the pain metric is a subjective patient opinion.

Aspect 9: The method of any one of aspects 4-8, wherein changing the at least one of the pulse duration, the frequency, or the amplitude of the transdermal stimulation routine comprises changing the at least one of the pulse duration, the frequency, or the amplitude based on a machine learning algorithm.

Aspect 10: The method of aspect 9, wherein the machine learning algorithm comprises a support vector machine.

Aspect 11: The method of any one of aspects 4-10, further comprising moving at least one electrode from a first location on the patient to a second location on the patient.

Aspect 12: The method of any one of aspects 4-11, wherein the stimulation routine comprises a plurality of repetitions, each repetition having a pulse width, each repetition comprising a plurality of frequency modulated pulses provided at a carrier frequency, wherein the repetitions are repeated at a repetition frequency.

Aspect 13: A method of treating pain in a patient, the method comprising:
applying, transdermally, by the first electrode, a stimulation routine to a portion of a spinal cord; and
increasing an intensity of the stimulation routine to a first threshold.

Aspect 14: The method of aspect 13, further comprising:
applying the stimulation routine at the first threshold during a session.

Aspect 15: The method of aspect 14, wherein applying the stimulation routine at the first threshold during the session comprises applying the stimulation routine at the first threshold for an entirety of the session.

Aspect 16: The method of aspect 14, wherein applying the stimulation routine at the first threshold during the session comprises intermittently reducing the stimulation routine below the first threshold.

Aspect 17: The method of any one of aspects 13-16, wherein the first threshold is determined by a patient tolerance.

Aspect 18: The method of any one of aspects 13-17, wherein the stimulation routine is a first stimulation, the method further comprising:
applying, transdermally, by the second electrode, a second stimulation routine to a spinal cord; and
increasing an intensity of the second stimulation routine by the second electrode to a second threshold.

Aspect 19: The method of aspect 18, further comprising:
applying the first stimulation routine by the first electrode at the first threshold during a session; and
applying the second stimulation routine by the second electrode at the second threshold during the session.

Aspect 20: The method of any one of aspects 13-19, wherein the stimulation routine comprises a plurality of repetitions of frequency modulated pulses, each repetition having a pulse width, wherein the plurality of repetitions are repeated at a repetition frequency.

Aspect 21: The method of any one of aspects 13-20, wherein the patient does not have a spinal cord injury that affects motor function.

Aspect 22: The method of aspect 21, wherein the patient does not have a spinal cord injury.

Aspect 23: The method of any one of aspects 13-22, further comprising repeating application of the stimulation routine for a plurality of sessions.

Aspect 24: The method of any one of aspects 13-23, wherein the stimulation routine remains constant for each session of the plurality of sessions.

Aspect 25: The method of aspect 23 or aspect 24, further comprising ceasing provision of additional sessions after pain is reduced by a threshold.

Aspect 26: The method of any one of aspects 23-25, further comprising ceasing provision of additional sessions upon absence of improvement of pain experience after one or more previous sessions.

Aspect 27: The method of any one of aspects 13-26, further comprising, prior to increasing the intensity of the stimulation routine to the first threshold, increasing the intensity of the stimulation to a baseline threshold at which the patient can feel the stimulation routine at the skin.

Aspect 28: The method of any one of aspects 13-27, wherein the first threshold is selected as a maximum tolerable level.

Aspect 29: A method comprising:
performing physical therapy during or after receipt of transdermal stimulation routine to a portion of a spinal cord to treat pain while experiencing reduced pain from the transdermal stimulation routine.

Although the foregoing invention has been described in some detail by way of illustration and example for purposes of clarity of understanding, certain changes and modifications may be practiced within the scope of the appended claims.

1. A method of treating pain in a patient, the method comprising:
applying a transdermal stimulation routine to a portion of a spinal cord to treat pain.

2. The method of claim 1, wherein the patient does not have a spinal cord injury that affects motor function.

3. (canceled)

4. The method of claim 1, further comprising determining the transdermal stimulation routine for treating pain in the patient, wherein determining the transdermal stimulation routine comprises changing at least one of a pulse duration, a frequency or an amplitude of the transdermal stimulation routine based on a pain metric.

5. The method of claim 4, wherein the pain metric is an EEG measurement.

6. The method of claim 4, wherein the pain metric is a blood biomarker.

7. The method of claim 6, wherein the biomarker is RNA.

8. (canceled)

9. The method of claim 4, wherein changing the at least one of the pulse duration, the frequency, or the amplitude of the transdermal stimulation routine comprises changing the at least one of the pulse duration, the frequency, or the amplitude based on a machine learning algorithm.

10. The method of claim 9, wherein the machine learning algorithm comprises a support vector machine.

11. (canceled)

12. The method of claim 4, wherein the stimulation routine comprises a plurality of repetitions, each repetition having a pulse width, each repetition comprising a plurality of frequency modulated pulses provided at a carrier frequency, wherein the repetitions are repeated at a repetition frequency.

13. A method of treating pain in a patient, the method comprising:
applying, transdermally, by a first electrode, a stimulation routine to a portion of a spinal cord of the patient;
increasing an intensity of the stimulation routine to a first threshold; and
applying the stimulation routine at the first threshold during a session.

14. (canceled)

15. The method of claim 13, wherein applying the stimulation routine at the first threshold during the session comprises applying the stimulation routine at the first threshold for an entirety of the session.

16. The method of claim 13, wherein applying the stimulation routine at the first threshold during the session comprises intermittently reducing the stimulation routine below the first threshold.

17. The method of claim 13, wherein the first threshold is determined by a patient tolerance.

18. The method of claim 13, wherein the stimulation routine is a first stimulation, the method further comprising:
applying, transdermally, by a second electrode, a second stimulation routine to a spinal cord;
increasing an intensity of the second stimulation routine by the second electrode to a second threshold; and
applying the second stimulation routine by the second electrode at the second threshold during the session.

19. (canceled)

20. The method of claim 13, wherein the stimulation routine comprises a plurality of repetitions of frequency modulated pulses, each repetition having a pulse width, wherein the plurality of repetitions are repeated at a repetition frequency.

21. The method of claim 13, wherein the patient does not have a spinal cord injury that affects motor function.

22. (canceled)

23. The method of claim 13, further comprising repeating application of the stimulation routine for a plurality of sessions.

24. (canceled)

25. The method of claim 23, further comprising ceasing provision of additional sessions after pain is reduced by a threshold.

26. The method of claim 23, further comprising ceasing provision of additional sessions upon absence of improvement of pain experience after one or more previous sessions.

27. The method of claim 13, further comprising, prior to increasing the intensity of the stimulation routine to the first threshold, increasing the intensity of the stimulation to a baseline threshold at which the patient can feel the stimulation routine at the skin.

28. (canceled)

29. (canceled)
